(19) United States
(12) Patent Application Publication
(10) Pub. No.: US 2024/0374489 A1
(43) Pub. Date: Nov. 14, 2024

(54) GLASS COMPOSITION

(71) Applicant: IR Scientific Inc., Halifax (CA)

(72) Inventors: Daniel BOYD, Upper Tantallon (CA); Kathleen O'CONNELL, South Bar (CA); Kathleen Naomi MACDONALD-PARSONS, Bedford (CA)

(21) Appl. No.: 18/784,024

(22) Filed: Jul. 25, 2024

Related U.S. Application Data
(63) Continuation of application No. 17/273,661, filed on Mar. 4, 2021, now Pat. No. 12,076,424.
(60) Provisional application No. 62/847,807, filed on May 14, 2019.
(60) Provisional application No. 62/727,377, filed on Sep. 5, 2018.

(51) Int. Cl. A61K 8/21; A61K 6/30; A61K 8/04; A61K 8/27; A61Q 11/00; C03C 3/14; C03C 3/23; C03C 4/00; C03C 12/00

(52) U.S. Cl. CPC A61K 8/21; A61K 6/30; A61K 8/042; A61K 8/27; A61Q 11/00; C03C 3/14; C03C 3/23; C03C 4/0021; C03C 12/00; C03C 2204/00

(57) ABSTRACT

The present disclosure provides a glass composition that include from about 50 mol % to about 95 mol % of B₂O₃; from about 5 mol % to about 50 mol % of one or more glass components selected from the group consisting of: Li₂O, Rb₂O, K₂O, Na₂O, SrO, CaO, MgO, and ZnO. The glass composition includes less than 30 mol % of Rb₂O. The glass composition is a quaternary system. The glass composition is a particulate material that includes particles that are from about 1 to about 50 μm in size. The glass composition loses at least 5 mass % within 24 hours when exposed to a buffered saline solution. The glass composition may be used to desensitize dentin. The present disclosure also provides a dentin-desensitizing composition.

### CROSS-REFERENCE TO RELATED APPLICATIONS

This application is a continuation of U.S. patent application Ser. No. 17/273,661, filed Mar. 4, 2021, which is a national phase entry of PCT/CA2019/051237, filed Sep. 4, 2019, which claims the benefit of U.S. Provisional Patent Application No. 62/727,377, filed Sep. 5, 2018; and of U.S. Provisional Patent Application No. 62/847,807, filed May 14, 2019, which are incorporated by reference herein in their entirety.

### FIELD

The present disclosure relates to glass compositions for dentin-desensitizing compositions.

### BACKGROUND

The following paragraphs are not an admission that anything discussed in them is prior art or part of the knowledge of persons skilled in the art.

Dentin sensitivity is dental pain that arises from exposed dentin surfaces in response to stimuli, such as thermal, evaporative, tactile, osmotic, chemical or electrical. Dentin sensitivity may be caused by gingival recession (receding gums) with exposure of root surfaces, loss of the cementum layer and smear layer, tooth wear, acid erosion, periodontal root planing, or dental bleaching.

Dentine contains many thousands of microscopic tubular structures that radiate outwards from the pulp. Changes in the flow of the plasma-like biological fluid present in the dentinal tubules can trigger mechanoreceptors present on nerves located at the pulpal aspect, thereby eliciting a pain response. This hydrodynamic flow can be increased by cold, air pressure, drying, sugar, sour (dehydrating chemicals), or forces acting onto the tooth. Hot or cold food or drinks, and physical pressure are typical triggers in those individuals with teeth sensitivity.

There is no universally accepted, gold-standard treatment which reliably relieves the pain of dental hypersensitivity in the long term. However, treatments can be divided into in-office (i.e. intended to be applied by a dentist or dental therapist), or treatments which can be carried out at home, available over-the-counter or by prescription.

The purported mechanism of action of these treatments is either occlusion of dentin tubules, or desensitization of nerve fibres/blocking the neural transmission.

### INTRODUCTION

The following introduction is intended to introduce the reader to this specification but not to define any invention. One or more inventions may reside in a combination or sub-combination of the apparatus elements or method steps described below or in other parts of this document. The inventors do not waive or disclaim their rights to any invention or inventions disclosed in this specification merely by not describing such other invention or inventions in the claims.

One example of a dentin-desensitizing composition known in the art is disclosed in PCT Publication No. WO2007144662A1. The disclosed toothpaste includes a bioactive glass comprising strontium. The disclosed bioactive glass occludes dentin tubules and induces precipitation and crystallisation of a carbonated hydroxyapatite. The disclosed bioactive glass is designed to degrade at the same rate as the rate of the induced tissue ingrowth.

One example of a dentin-desensitizing composition known in the art is disclosed in U.S. Pat. No. 5,735,942. The disclosed toothpaste includes a mineral composition composed of CaO, Na₂O, P₂O₅ and SiO₂. The disclosed mineral composition chemically reacts with the surface of dentin and intimately bonds to tooth structure.

One or more described embodiments attempt to address or ameliorate one or more shortcomings involved with dentin-desensitizing compositions that include non-degradable particulate material that occludes dentin tubules. In some embodiments, the disclosed particulate material substantially degrades over a period between 12 and 24 hours under environmental conditions. In some embodiments, the disclosed particulate material provides a controlled release of fluoride over the same time period.

In some embodiments, the present disclosure provides a glass composition that includes from about 50 mol % to about 95 mol % of B₂O₃; from about 5 mol % to about 50 mol % of a glass component selected from the group consisting of: Li₂O, Rb₂O, K₂O, Na₂O, SrO, CaO, MgO, ZnO and any combination thereof; 0 mol % of CuO; less than 0.1 mol % of BaO; and less than 0.1 mol % of P₂O₅; where the glass composition comprises less than 30 mol % of Rb₂O. The glass composition loses at least 5 mass % within 24 hours when exposed to a buffered saline solution, and the glass composition is a particulate material that comprises particles that are from about 1 to about 50 μm in size. The glass composition does not consist solely of B₂O₃ and Na₂O.

In some examples of glass compositions according to the present disclosure, less than 20 mol %, such as less than 15 mol %, less than 10 mol %, or less than 5 mol % of the glass composition is CaO, MgO, and Na₂O.

The glass composition may additionally include up to about 30 mol % of fluoride, wherein the fluoride is in the form of: CaF₂, NaF, Na₂PO₃F, KF, or SnF₂.

In other embodiments, the present disclosure provides a glass composition for desensitizing dentin, the glass composition includes from about 50 mol % to about 95 mol % of B₂O₃; from about 5 mol % to about 50 mol % of a glass component selected from the group consisting of: Li₂O, Rb₂O, K₂O, Na₂O, SrO, CaO, MgO, ZnO and any combination thereof; where the glass composition comprises less than 30 mol % of Rb₂O. The glass composition loses at least 5 mass % within 24 hours when exposed to a buffered saline solution, and the glass composition is a particulate material that comprises particles that are from about 1 to about 50 μm in size.

In some examples of the glass compositions, less than 20 mol %, such as less than 15 mol %, less than 10 mol %, or less than 5 mol % of the glass composition is CaO, MgO, and Na₂O.

The glass composition may additionally include up to about 30 mol % of fluoride, wherein the fluoride is in the form of: CaF₂, NaF, Na₂PO₃F, KF, or SnF₂.

In other embodiments, the present disclosure provides a glass composition that includes: fluoride, which is provided as from about 5 mol % to about 10 mol % of CaF2, SnF₂, NaF, KF, or any combination thereof; and from about 90 mol % to about 95 mol % of a combination of B₂O₃, Na₂O, MgO, and CaO, where the boron, the magnesium, the combination of Na and any K, and the combination of Ca and any Sn in the glass composition are present in elemental ratios of about 20: about 4: about 6: about 3, respectively.

One example of such a specific glass composition according to the present disclosure includes: about 50 mol % B₂O₃, about 15 mol % Na₂O, about 20 mol % MgO, about 10 mol % CaO, and about 5 mol % CaF₂.

Glass compositions according to the present disclosure may be formulated into a dentin-desensitizing composition, such as a toothpaste, a prophylaxis paste, a tooth varnish, a mouthwash, a dental gel, or a bonding agent. Dentin-desensitizing compositions according to the present disclosure are substantially water-free.

Glass compositions according to the present disclosure may be used for desensitizing dentin, such as in methods that include applying to an individual's dentin: a toothpaste, a prophylaxis paste, a tooth varnish, a mouthwash, a dental gel, or a bonding agent according to the present disclosure.

A glass composition according to the present disclosure may be prepared from a corresponding bulk glass. The chemical formulations are the same between the bulk glass and the particulate material. Another aspect of the present disclosure is a bulk glass having a chemical formulation as disclosed herein.

### BRIEF DESCRIPTION OF THE DRAWINGS

Embodiments of the present disclosure will now be described, by way of example only, with reference to the attached Figures.

FIG. 1 is an illustration of the average of the surface profile of a resin composite after 20,000 brushing cycles with Gel 7 HT toothpaste (“Gel”) vs. Gel 7 HT toothpaste formulated with a glass composition according to the present disclosure (“Gel+Additive”).

FIG. 2 is an illustration of the average of the surface profile of a resin composite after 20,000 brushing cycles with Gel 7 HT (“Gel”) toothpaste vs. Colgate™ Enamel Health Sensitivity Relief™ toothpaste formulated with the glass composition (“Colgate EN+Additive”).

FIG. 3 is an illustration of the average of the surface profile of a resin composite after 20,000 brushing cycles with Gel 7 HT toothpaste (“Gel”) vs. Colgate™ Optic White™ toothpaste (“Colgate Optic”).

FIG. 4 is an illustration of the average of the surface profile of a resin composite after 20,000 brushing cycles with Gel 7 HT (“Gel”) toothpaste vs. Colgate™ Enamel Health Sensitivity Relief™ toothpaste (“Colgate EN”).

FIG. 5 is an illustration of the average of the surface profile of a resin composite after 20,000 brushing cycles with Gel 7 HT toothpaste (“Gel”) vs. Sensodyne™ Whitening Repair and Protect™ toothpaste (“Sensodyne”).

FIG. 6 is an illustration of the average of the surface profile of an enamel surface after 20,000 brushing cycles with Gel 7 HT toothpaste formulated with the glass composition (“Gel+Additive”) vs. Sensodyne™ Whitening Repair and Protect™ toothpaste (“Sensodyne”).

FIG. 7 is an illustration of the average of the surface profile of an enamel surface after 20,000 brushing cycles with Gel 7 HT toothpaste formulated with the glass composition (“Gel+Additive”) vs. Colgate™ Enamel Health Sensitivity Relief™ toothpaste formulated with the glass composition (“Colgate EN+Additive”).

FIG. 8 is an illustration of the average of the surface profile of an enamel surface after 20,000 brushing cycles with Gel 7 HT toothpaste (“Gel”) vs. Gel 7 HT toothpaste formulated with the glass composition (“Gel+Additive”).

FIG. 9 is an illustration of the average of the surface profile of an enamel surface after 20,000 brushing cycles with Gel 7 HT toothpaste formulated with the glass composition (“Gel+Additive”) vs. Colgate™ Optic White™ toothpaste (“Colgate Optic”).

FIG. 10 is an illustration of the average of the surface profile of an enamel surface after 20,000 brushing cycles with Gel 7 HT toothpaste formulated with the glass composition (“Gel+Additive”) vs. Colgate™ Enamel Health Sensitivity Relief™ toothpaste (“Colgate EN”).

FIG. 11 is an image from a scanning electron microscope of an exemplary glass composition according to the present disclosure after 30 minutes in simulated body fluid (SBF) at 37° C.

FIG. 12 is an image from a scanning electron microscope of an exemplary glass composition according to the present disclosure after 3 hours in simulated body fluid (SBF) at 37° C.

FIG. 13 is an image from a scanning electron microscope of an exemplary glass composition according to the present disclosure after 12 hours in simulated body fluid (SBF) at 37° C.

FIG. 14 is another photograph from a scanning electron microscope of an exemplary glass composition according to the present disclosure after 12 hours in simulated body fluid (SBF) at 37° C.

### DETAILED DESCRIPTION

Glass compositions according to the present disclosure are at least a quaternary system. The glass compositions include from about 50 mol % to about 95 mol % of B₂O₃; and from about 5 mol % to about 50 mol % of one or more glass components selected from the group consisting of: Li₂O, Rb₂O, K₂O, Na₂O, SrO, CaO, MgO, and ZnO. The glass compositions include less than 30 mol % of Rb₂O. Glass compositions according to the present disclosure degrade under physiological conditions, losing at least 5 mass % within 24 hours when exposed to a buffered saline solution.

The glass composition is a particulate material that includes particles that are from about 1 to about 50 μm in size. The glass composition includes at least some particles that are sized to occlude dentinal tubules, thereby desensitizing the dentin. In the context of the present disclosure, a particle sized to occlude a dentinal tubule should be understood to mean that the particle sits in or on top of the dentinal tubule, reducing the movement of the dentinal fluid.

In the context of the present disclosure, a glass composition that is “at least a quaternary system” should be understood to refer to glasses with four or more different elements. For example, a glass composition that is made up of only B₂O₃, Li₂O and ZnO would be considered a quaternary system since the glass includes the elements boron, lithium, zinc, and oxygen. Similarly, a glass composition that is made up of only B₂O₃, CaO and CaF₂ would be considered a quaternary system since the glass includes the elements boron, calcium, fluorine, and oxygen. In contrast, a glass composition that is made up of only B₂O₃ and Na₂O would be considered a ternary system since the glass includes the elements boron, sodium, and oxygen.

It should be understood that “about 5 mol % to about 50 mol % of one or more glass components” refers to the total mol % of the glass components, and does not refer to the mol % percent of each individual component. For example, a glass composition according to the present disclosure could include 2.5 mol % of Li₂O and 2.5 mol % of ZnO in order to provide the recited 5 mol % of the additional glass components.

It should be understood that “about X mol %” refers to any value that is within ±2% of the reported percentage. For example, “about 10 mol %” would refer to values from 8 mol % to 12 mol % since all those values would be within ±2% of the reported 10%; and “about 50 mol %” would refer to values from 48 mol % to 52 mol % since all those values would be within ±2% of the reported 50%.

It should be understood that “about X μm” in the context of particle size is determined based on accepted tolerances as per ASTM for a test sieve of the noted size. For example, the accepted tolerance for a 50 μm test sieve is 3 μm. Accordingly, “about 50 μm” refers to particles that are from 47 μm to 53 μm in size. In another example, the accepted tolerance for a 35 μm test sieve is 2.6 μm. Accordingly, “about 35 μm” refers to particles that are from 32.4 μm to 38.6 μm in size. The ASTM accepted tolerance for a 25 μm sieve is 2.2 μm. For test sieves without a standard, accepted tolerance (such as test sieves below 20 μm), the expression “about X μm” refers to ±15% for sizes from 5 to 15 μm, and ±50% for sizes less than 5 μm. For example “about 1 μm” refers to particles that are from 0.5 to 1.5 μm in size.

### Glass Compositions

Glass compositions according to the present disclosure may include a source of fluoride, such as CaF₂, NaF, Na₂PO₃F, KF, or SnF₂. Including fluoride in the glass composition results in fluoride being released when the glass degrades. The released fluoride may form fluorapatite (Ca₅(PO₄)₃F) in or around the dentinal tubules, which may form a protective precipitate and further decrease dentin sensitivity. In a glass composition that includes fluoride the source of fluoride may be up to 30 mol % of the glass composition. In some examples, the source of fluoride may be from about 1 mol % to about 10 mol %, for example from about 1 mol % to about 5 mol %, of the glass composition. In particular examples, the source of fluoride is about 15 mol % of the composition. Compositions that include CaF₂ or SnF₂ provide twice the amount of fluoride per mole of starting material compared to compositions that use NaF, Na₂PO₃F, or KF.

In some examples, the glass composition includes from about 1 mol % to about 10 mol % of fluoride. In some examples, the glass composition includes from about 1 mol % to about 5 mol % of fluoride.

In some examples, glass composition includes sufficient fluoride that 0.1 g of the particulate material releases the fluoride into 10 ml of a buffered saline solution at an average rate of about 1 ppm/hr to about 15 ppm/hr over 1, 2, 4, 8, 12, 18 or 24 hours. In the context of the present disclosure, ppm is measured as mass/mass. In particular examples, the glass composition includes sufficient fluoride that about 4 to about 6 ppm of fluoride is released per hour over 1 hour.

In some examples of glass compositions according to the present disclosure, less than 20 mol %, such as less than 15 mol %, less than 10 mol %, or less than 5 mol % of the glass composition is CaO, MgO, and Na₂O.

In an example of a glass composition according to the present disclosure, the glass composition does not include any CuO; and includes less than 0.1 mol % of BaO, and less than 0.1 mol % of P₂O₅. In particular examples, the glass composition does not include any CuO, BaO, or P₂O₅.

A glass composition according to the present disclosure may include from about 5 mol % to about 50 mol % of one or more glass components selected from the group consisting of: Li₂O, Rb₂O, K₂O, Na₂O, SrO, and ZnO; and wherein the glass composition comprises less than 0.1 mol % of CaO and less than 0.1 mol % of MgO.

A glass composition according to the present disclosure may include from about 5 mol % to about 50 mol % of one or more glass components selected from the group consisting of: Li₂O, Rb₂O, K₂O, SrO, and ZnO; and wherein the glass composition comprises less than 0.1 mol % of CaO, less than 0.1 mol % of MgO, and less than 0.1 mol % of Na₂O.

A glass composition according to the present disclosure may include from about 50 mol % to about 80 mol % of B₂O₃, such as about 50 mol % of B₂O₃.

A glass composition according to the present disclosure may include about 5 mol % to about 40 mol %, such as from about 20 mol % to about 40 mol %, of the one or more glass components selected from the group consisting of: Li₂O, Rb₂O, K₂O, Na₂O, SrO, CaO, MgO, and ZnO.

A glass composition according to the present disclosure may include B₂O₃, Li₂O, and ZnO, and optionally Rb₂O, Na₂O, and/or a source of fluoride. In particular examples, the glass composition includes: from about 5 mol % to about 25 mol % Li₂O, and from about 5 mol % to about 25 mol % Rb₂O; or from about 5 mol % to about 25 mol % Li₂O, and from about 5 mol % to about 15 mol % ZnO, and optionally from about 5 mol % to about 15 mol % of Na₂O. The glass compositions may include about 50 mol % of B₂O₃, or about 70 mol % B₂O₃.

A glass composition according to the present disclosure may include B₂O₃, and ZnO, and optionally Rb₂O and/or a source of fluoride. In particular examples, the glass composition includes: from about 5 mol % to about 30 mol % ZnO. If present, the RbO₂ may be included in an amount from about 5 mol % to about 30 mol %. The glass compositions may include about 50 mol % of B₂O₃.

A glass composition according to the present disclosure may include B₂O₃, and SrO, and optionally ZnO and/or a source of fluoride. In particular examples, the glass composition includes: from about 5 mol % to about 30 mol % SrO. If present, the ZnO may be included in an amount from about 5 mol % to about 30 mol %. The glass compositions may include about 50 mol % of B₂O₃.

As noted above, the present disclosure also provides a glass composition 25 that includes: fluoride, which is provided as from about 5 mol % to about 10 mol % of CaF₂, SnF₂, NaF, KF, or any combination thereof; and from about 90 mol % to about 95 mol % of a combination of B₂O₃, Na₂O, MgO, and CaO, where the boron, the magnesium, the combination of Na and any K, and the combination of Ca and any Sn in the glass composition are present in elemental ratios of about 20: about 4: about 6: about 3, respectively.

One specific example of such a glass composition includes: about 50 mol % B₂O₃, about 15 mol % Na₂O, about 20 mol % MgO, about 10 mol % CaO, and about 5 mol % CaF₂. This composition may be referred to herein as composition “PBF1”.

Another specific example of such a glass composition includes: about 48 mol % B₂O₃, about 9 mol % Na₂O, about 19 mol % MgO, about 14 mol % CaO, and about 10 mol % NaF. This composition may be referred to herein as composition “PBF1-Na”.

### Particle Size Distribution

A glass composition according to the present disclosure is a particulate material that includes particles that are from about 1 to about 50 μm in size. At least some of the particles are sized to sit in or on top of a dentinal tubule. Dentinal tubules have a natural variation in diameter and are primarily from about 0.5 to about 8 μm in size, for example, from about 0.5 to about 5 μm in size. Accordingly, glass compositions of the present disclosure may be used for desensitizing dentin, which may temporarily reduce pain associated with sensitive teeth.

In some examples, at least 75% of the particles making up the particulate material are smaller than 50 μm in size. In other examples, at least 85% or at least 95% of the particles are smaller than 50 μm in size. In some examples, at least 5% of the particles making up the particulate material are smaller than 7 μm in size.

In particular examples, the particulate material is made up of a plurality of particles where at least 5% of the particles are smaller than 35 μm in size, at least 5% of the particles are smaller than 15 μm in size, and at least 5% of the particles are smaller than 7 μm in size.

In particular examples, the particulate material is made up of a plurality of particles where at least 5% of the particles are from about 15 μm to about 35 μm in size, at least 5% of the particles are from about 6 μm to about 15 μm in size, and at least 5% of the particles are from about 3 μm to about 7 μm in size.

In some particular examples, the particulate material is made up of a plurality of particles where the particle size distribution is Dx10 of about 5 um, Dx50 of about 15 um, and Dx90 of about 30 um.

### Degradation

Glass compositions according to the present disclosure degrade under physiological conditions, losing at least 5 mass % within 24 hours when exposed to a buffered saline solution. In some examples, the glass composition may lose at least 20 mass %, at least 40 mass %, at least 60 mass %, or at least 80 mass % within 24 hours when exposed to the buffered saline solution.

### Dentin-Desensitizing Compositions

Glass compositions according to the present disclosure may be formulated in a dentin-desensitizing composition that includes a water-free, orally-compatible carrier. Dentin-desensitizing compositions according to the present disclosure are free of water since the glass composition degrades if exposed to water.

In the context of the present disclosure, “water-free” should be understood to mean that the dentin-desensitizing composition includes so little water that the glass composition remains capable of reducing dentin sensitivity over the expected lifespan of the product. The expected lifespan of the product refers to the longest expected time between when the dentin-desensitizing composition was produced and when the dentin-desensitizing composition was completely used up or disposed of.

The orally-compatible carrier used in the dentin-desensitizing composition may be a mouthwash, a carrier formulated to mix with additional components to form a mouthwash, or an orally-compatible viscous carrier, such as a toothpaste, a dental gel, a prophylaxis paste, a tooth varnish, a bonding agent, or a carrier that is formulated to mix with additional components to form a toothpaste. The orally-compatible viscous carrier may have a viscosity from about 100 cP at 30° C. to about 150,000 cp at 30° C.

The dentin-desensitizing composition may include a glass composition according to the present disclosure that includes fluoride, as discussed above, where the glass composition is present in a sufficient amount that the desensitizing composition includes about 100 ppm to about 5,000 ppm of the fluoride.

One example of a dentin-desensitizing composition according to the present disclosure is a toothpaste that includes a glass composition according to the present disclosure and: an abrasive; a detergent such as sodium lauryl sulfate; a fluoride source; an antibacterial agent; a flavorant; a remineralizer; a sugar alcohol such as glycerol, sorbitol, or xylitol; another dentin desensitizing agent; a hydrophilic polymer such as polyethylene glycol; or any combination thereof. The glass composition may be from about 0.5 to about 15 mass % of the toothpaste.

One particular example of a dentin-desensitizing composition according to the present disclosure is a toothpaste that includes a glass composition according to the present disclosure and: glycerin, silica, a polyethylene glycol (such as PEG 400), titanium dioxide, a carbomer, and a sweetener (such as potassium acesulfame or sodium saccharin).

Another particular example of a dentin-desensitizing composition according to the present disclosure is a toothpaste that includes a glass composition according to the present disclosure and: α-carbomer, DL-limonene, glycerin, mint flavor, a polyethylene glycol (such as PEG-8), silica, titanium dioxide, sodium lauryl sulphate, and a sweetener (such as potassium acesulfame or sodium saccharin).

Another example of a dentin-desensitizing composition according to the present disclosure is a carrier that includes a glass composition according to the present disclosure, where the carrier is formulated to be mixed with additional components to form a toothpaste.

Yet another example of a dentin-desensitizing composition according to the present disclosure is a carrier formulated to mix with additional components to form a mouthwash. Particular examples of the carrier include a glass composition according to the present disclosure and: a water-free alcohol, cetylpyridinium chloride, chlorhexidine, an essential oil, benzoic acid, a poloxamer, sodium benzoate, a flavor, a coloring, or any combination thereof. The additional component(s) that is/are mixed with the carrier to form the mouthwash may include: water, peroxide, cetylpyridinium chloride, chlorhexidine, an essential oil, alcohol, benzoic acid, a poloxamer, sodium benzoate, a flavouring, a colouring, or any combination thereof. The carrier and the additional components may be kept in separate compartments, and mixed together before the mixture is used as a mouthwash. The separate compartments may be in the form of a multi-chambered bottle, such as a bifurcated bottle.

Another example of a dentin-desensitizing composition according to the present disclosure is a prophylaxis paste (also referred to as a “prophy paste”) that includes a glass composition according to the present disclosure. Particular examples of contemplated prophy pastes include a glass composition according to the present disclosure and: pumice, glycerin, diatomite (preferably fine grit), sodium silicate, methyl salicylate, monosodium phosphate, sodium carboxymethylcellulose, a sweetener (such as potassium acesulfame or sodium saccharin), a flavouring, a colouring, or any combination thereof.

### Methods

Glass compositions according to the present disclosure may be synthesized by: mixing appropriate molar amounts of the starting reagents; packing the precursor blend in a platinum crucible (Johnson Matthey, Noble Metals, Pennsylvania); placing the packed crucible in a furnace (Carbolite, RHF 1600) at room temperature; heating the furnace (such as at a rate of 25° C./minute) to an initial dwelling temperature of 600° C.; holding the temperature for 60 minutes; ramping the temperature (such as at a rate of 20° C./minute) to a dwelling temperature of 1,100° C.; holding the temperature for 60 minutes; and quenching the glass melt between two stainless steel plates.

It should be understood that the specific ramp rate, times, and temperatures disclosed above could be modified, so long as the glass melts. Ramp rates from 10-20 degrees/min, and holding at the dwell temperature may remove at least some gas bubbles from the glass.

The resulting quenched glasses may be ground/milled separately within a planetary micro mill (Pulverisette 7, Fritsch, Germany) and sieved with ASTM E-11 compliant sieves (Cole Palmer, U.S.A) to obtain particles of <25 μm. Glasses may be stored under vacuum in glass scintillation vials.

Although the resulting glass composition includes oxides, the starting reagents may include oxides, carbonates, or both. For example, the starting reagent may include boron oxide, rubidium carbonate, lithium carbonate, and calcium fluoride. The rubidium carbonate and lithium carbonate decompose in the furnace to release CO₂, generating their corresponding oxides.

Particle size is measured using a Malvern Mastersizer (MS) 3000 laser diffraction particle size analyzer. Glass powders are separately suspended in deionized water to obtain an obscuration value for the suspension between 5-8%. Suspensions are measured using both a blue (λ=470 nm) and red (λ=632.8 nm) laser and are measured 5 times (n=5).

Fluoride release is measured by placing 0.1 g of the glass composition in 10 ml of TRIS buffered saline (BioUltra, Sigma Aldrich, Canada) in a 15 ml Falcon tube. The solution is agitated at 120 rpm and kept at a temperature of 37° C. for the desired release period, such as for 1, 3, 6, 12 or 24 h. On completion, the liquid portion is decanted and filtered using a 0.22 μm filter (Sarstedt syringe filter, Canada) into new clean 15 ml Falcon tubes, which were capped and then stored at 4° C. until the amount of fluoride is quantified. The concentration of the released fluoride is quantified using an Accumet® AB250 pH/ion selective electrode meter equipped with electrode fluoride combination (Accumet®). Standard solutions are prepared using a fluoride analytical standard specifically for ion selective electrodes (NaF, 0.1 M F, Sigma Aldrich, Canada) and calibration cures are retrieved before analysis. Liquid extracts derived from the extraction of each composition were prepared for ion analysis as per the electrode manufactures instructions. The ion concentrations are reported as the average of n=3±SD.

In the context of the present disclosure, mass loss of a glass composition is in relation to a solid glass cylinder that is 6 mm in length and 4 mm in diameter. The glass cylinder is prepared by producing molten glass, as described above, quenching the molten glass in a stainless steel mould (6 mm in length by 4 mm in diameter), set between two stainless steel plates. The excess glass on the cylinders is carefully etched off by a Speedy Sharp utensil and the remainder of the excess glass is removed (while placed back into the stainless steel moulds) using a grinding/polishing wheel with 240 sand paper and applying pressure to the moulds/glass on the wheel. Glass cylinders with uneven edges, air bubbles, or chips are excluded.

The mass loss for a given glass composition is measured using three cylinders. The length and diameter of each cylinder is measured and recorded 3 times (changing the position of measurement each time) and recorded as an average±SD. The mass of each cylinder is measured separately (Sartorius Cubis, Model MSU-224S 100 DI, Cole Palmer). The three cylinders are placed in separate 50 mL falcon tubes with 20 mL of TRIS buffered saline (BioUltra, Sigma Aldrich, Canada) in each tube. The tubes are then placed in a shaking incubator (Thermos Scientific, MaxQ 4000) at 37° C. and agitated at 120 rpm for 24 hrs. After the 24 hours, the cylinders are filtered from the solution, rinsed with cold distilled H₂O, and placed to dry overnight in a 37° C. oven. Once dry, the length, diameter and mass are measured.

Abrasiveness of a composition is determined by measuring the gloss and surface roughness of a resin composite or an enamel surface after brushing with the composition. ESPE Filtek Supreme Ultra Universal Restorative, shade A2B (3M, St. Paul, Minnesota, USA), is cured in a 12.7 mm diameter, 2 mm thick metal split-mold. Mylar sheets are placed above and below the mold and glass plates are used to press the composite flat and squeeze out any extra material. A broadband multiwave LED light curing unit (Valo Grand, Ultradent Products, South Jordan, Utah, USA) is placed directly on top of the specimens and cured for 20 s at the standard setting. Excess material is removed by hand before mounting the specimens for brushing.

Samples are stored at 37° C. in the dark for a minimum of 24 h before use. The surface of the enamel specimens are prepared by polishing with varying levels of grit to produce a flat and smooth surface. Low grit sandpaper is used to create the initial flat surface (P800C, Klingspor, Haiger, Germany) and then increasing amounts of grit are used, followed by polishing. The final polishing steps are carried out on cloth pads with 3 μm and then 0.3 μm alumina oxide powder slurries (Buehler Ltd., Lake Bluff, Illinois, USA). Each polishing step is carried out for approximately 1 min with pressure applied by hand.

A custom-built brushing machine (Ultradent, South Jordan, UT, USA) simulates toothbrushing of 10 samples simultaneously. It is equipped with toothbrushes (GUM brand 459PC, Sunstar, Guelph, Ontario, Canada) with constant loads of 176 g applied during brushing. The toothbrushes are replaced after 10,000 brushing cycles. The samples are covered with a minimum of 3 mm of a 5:8 weight ratio toothpaste slurry with distilled water during brushing. Samples are rotated to a different position every 2,500 brush cycles (ensuring that the sample was brushed with the same toothpaste each time it was moved). 20,000 brush cycles represent approximately 2 years of brushing. The repetitions and position of the toothpastes in the machine are randomized with a random number generator (for each substrate material), while manually assuring there was a minimum of two repeats of the same toothpaste during a single run to ensure rotation of toothbrushes.

A glossmeter (Novo-Curve G, Rhopoint Instruments, Hastings, UK) is used to measure the gloss of the composite and enamel surfaces. The gloss is measured in three random points to create an average for the surface. The glossmeter calibration is verified each day using a traceable calibration tile with high and low reflectivity. The gloss is measured at 0, 5,000, 10,000, 15,000 and 20,000 brush cycles and new toothpaste slurry is used after measuring the gloss.

The average roughness of the surfaces is also measured before brushing and after 20,000 brushing cycles. An atomic force microscope (nGauge, ICSPI Corporation, Rev. 1.0, Waterloo, Ontario, Canada) is used to measure the average roughness at 3 different positions to form an average of the surface. A 25×25 μm area is scanned at a rate of 1200 μs/pixel. The data is analyzed using Gwyddion (http://gwyddion.net) software.

### EXAMPLES

The glass compositions shown in Table 1 were all synthesized by: weighing determined amounts of the analytical grade reagents (boron oxide, rubidium carbonate, lithium carbonate, and calcium fluoride) (Sigma Aldrich, Canada). The individual formulations were mixed for 60 mins to ensure homogeneity. Each precursor blend was placed and packed in 50 mL platinum crucibles (Johnson Matthey, Noble Metals, Pennsylvania). The pack crucible was then placed in a furnace (Carbolite, RHF 1600) at room temperature. The furnace was heated (25° C./minute) to an initial dwelling temperature of 600° C. and held for 60 minutes. The temperature was then ramped (20° C./minute) to a final dwelling temperature of 1,100° C. and held for 60 minutes. On removal, each glass melt was quenched between two stainless steel plates. The resulting quenched glasses were ground/milled separately within a planetary micro mill (Pulverisette 7, Fritsch, Germany) and sieved with ASTM E-11 compliant sieves (Cole Palmer, U.S.A) to obtain particles of <25 μm.

TABLE 1
Exemplary glass compositions
according to the present disclosure

| Glass | B₂O₃ | Li₂O | Rb₂O | CaF₂ |
|---|---|---|---|---|
| Identifier | (mol %) | (mol %) | (mol %) | (mol %) |
| BCF100 | 70 | 20 | 10 | 0 |
| BCF101 | 70 | 20 | 9 | 1 |
| BCF102 | 70 | 20 | 8 | 2 |
| BCF103 | 70 | 20 | 7 | 3 |
| BCF104 | 70 | 20 | 6 | 4 |
| BCF200 | 70 | 10 | 20 | 0 |
| BCF201 | 70 | 9 | 20 | 1 |
| BCF202 | 70 | 8 | 20 | 2 |
| BCF203 | 70 | 7 | 20 | 3 |
| BCF204 | 70 | 6 | 20 | 4 |

The particle size distribution for the exemplary glasses of Table 1 is shown in Table 2.

TABLE 2
Particle size distribution (μm).

| Glass | Dx | Dx | Dx |
|---|---|---|---|
| Identifier | (10) | (50) | (90) |
| BCF100 | 5.16 | 14.5 | 29.7 |
| BCF101 | 4.23 | 11.9 | 25 |
| BCF102 | 4.77 | 13.8 | 29.1 |
| BCF103 | 4.75 | 13.6 | 28.4 |
| BCF104 | 3.97 | 12.7 | 28.3 |
| BCF200 | 6.87 | 15.1 | 27.6 |
| BCF201 | 6.31 | 16.2 | 31.7 |
| BCF202 | 6.5 | 16.9 | 32.5 |
| BCF203 | 6.22 | 16.5 | 32.4 |
| BCF204 | 6.35 | 17 | 33.6 |

It should be understood that where “Dx(#)” refers to #% of particles that are smaller in size than the noted value. For example, BCF100 has a Dx(10) of 5.16 microns, which means that 10% of the particles are less than 5.16 microns in size.

The particles of the exemplary glasses of Table 1 were evaluated for fluoride release in a buffered saline solution over 12 and 24 hours using the method discuss above. The ppm values of released fluoride are shown in Table 3.

TABLE 3
Mean fluoride release (ppm)
at 12 and 24 hours.

| | Fluoride release- | Fluoride release- |
|---|---|---|
| Glass | 12 hours | 24 hours |
| Identifier | (ppm) | (ppm) |
| BCF100 | 0 | 0 |
| BCF101 | 32 | 31 |
| BCF102 | 59 | 58 |
| BCF103 | 69 | 77 |
| BCF104 | 65 | 70 |
| BCF200 | 0 | 0 |
| BCF201 | 24 | 23 |
| BCF202 | 48 | 47 |
| BCF203 | 62 | 59 |
| BCF204 | 76 | 71 |

BCF201 was formulated into two toothpastes to test the abrasive effects of the glass particles, and compared to the abrasive effects of Sensodyne™ Whitening Repair and Protect™ toothpaste (“Sensodyne”), and Colgate™ Optic White™ toothpaste (“Colgate Optic”). The exemplary glass was formulated with: (a) Colgate™ Enamel Health Sensitivity Relief™ (Colgate-Palmolive, Toronto, ON, Canada) (“Colgate EN”), or (b) Gel 7 HT (Germiphene, Brantford, ON, Canada) (“Gel”), a neutral pH fluoride gel dentifrice that does not contain any abrasive materials.

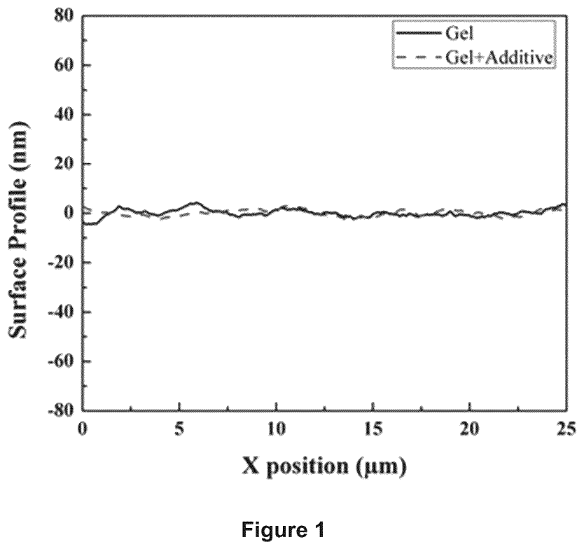

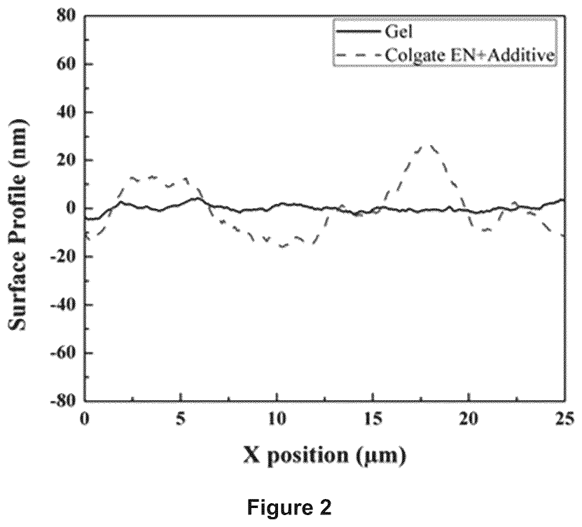

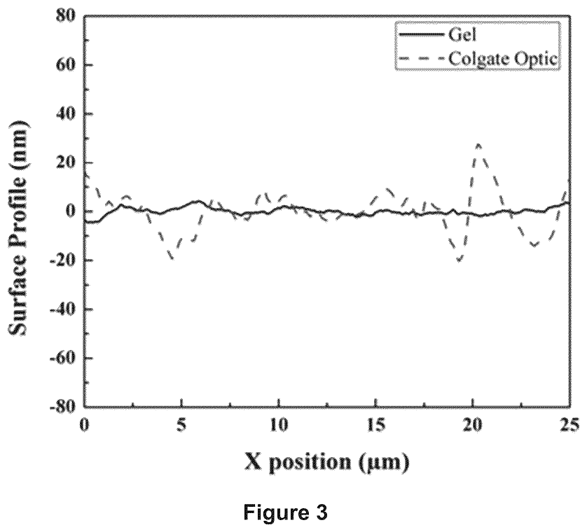

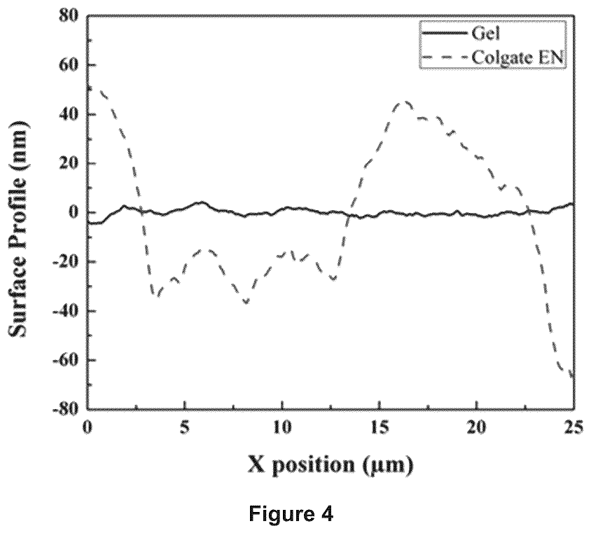

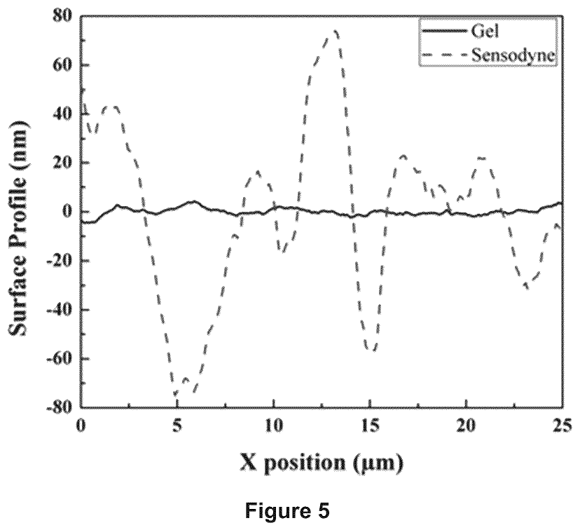

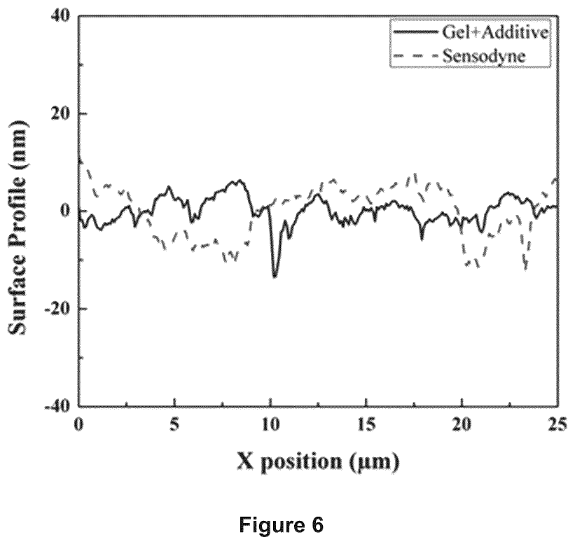

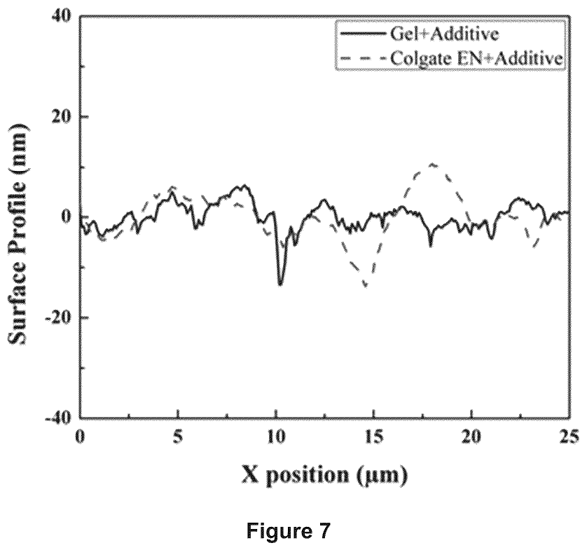

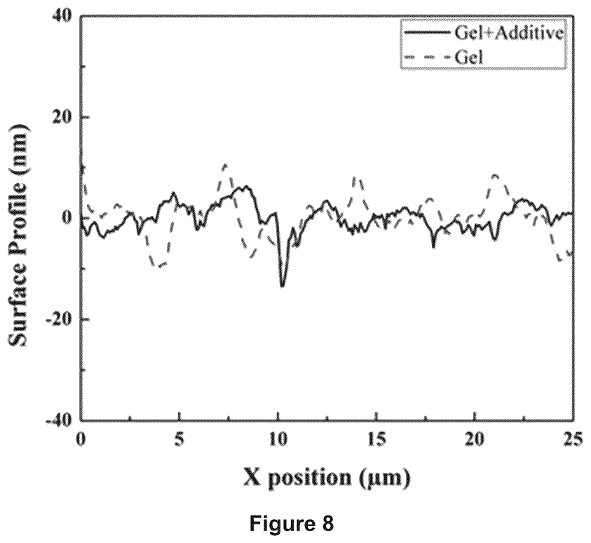

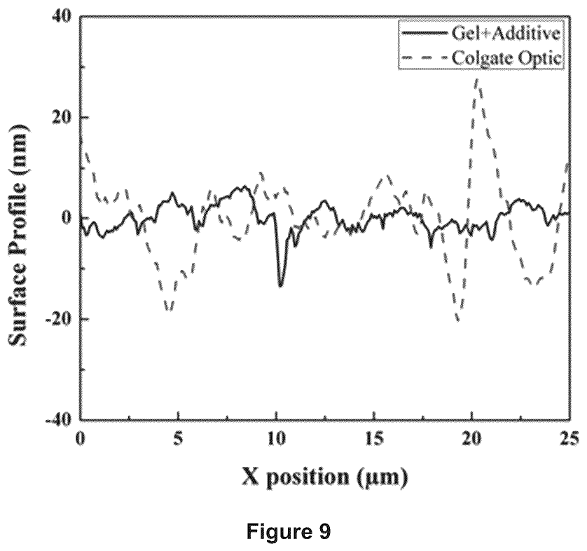

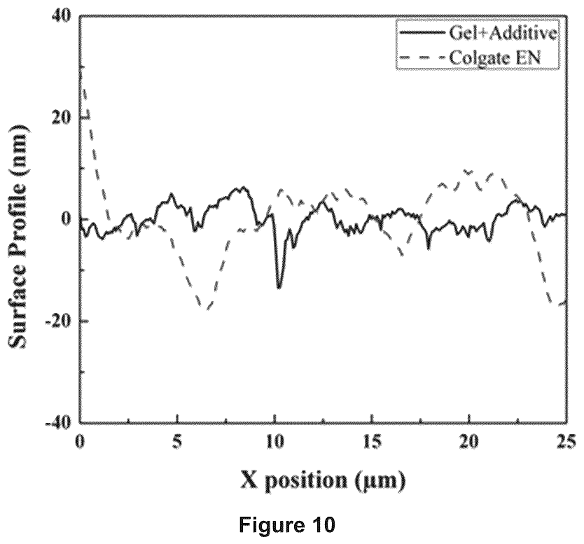

The results of the abrasive tests are illustrated the following tables, and in FIGS. 1 to 10. Table 4 shows the Gloss Units of a resin composite surface after different numbers of brushing cycles using the different toothpastes. Table 5 shows the Gloss Units of a resin composite after different numbers of brushing cycles using the different toothpastes. Table 6 shows the roughness of a resin composite surface after 20,000 brushing cycles using the different toothpastes. Table 7 shows the roughness of an enamel surface after 20,000 brushing cycles using the different toothpastes.

TABLE 4
Gloss Units of a resin composite surface
after different numbers of brushing cycles

| | | | | Colgate | | |
|---|---|---|---|---|---|---|
| Brushing | | Gel + | Colgate | EN + | Colgate | Senso- |
| cycles | Gel | BCF201 | EN | BCF201 | Optic | dyne |
| 0 | 91 +/− 3 | 91 +/− 3 | 90 +/− 3 | 90 +/− 3 | 90 +/− 3 | 91 +/− 3 |
| 5,000 | 91 +/− 4 | 91 +/− 3 | 63 +/− 9 | 69 +/− 8 | 75 +/− 6 | 56 +/− 8 |
| 10,000 | 90 +/− 3 | 91 +/− 3 | 55 +/− 1 | 64 +/− 10 | 68 +/− 8 | 46 +/− 9 |
| 15,000 | 90 +/− 3 | 91 +/− 3 | 49 +/− 7 | 59 +/− 9 | 62 +/− 8 | 39 +/− 8 |
| 20,000 | 89 +/− 4 | 90 +/− 3 | 46 +/− 6 | 52 +/− 8 | 56 +/− 10 | 25 +/− 7 |

TABLE 5
Gloss Units of an enamel surface after different numbers of brushing cycles

| Brushing | | Gel + | Colgate | Colgate | Colgate | |
|---|---|---|---|---|---|---|
| cycles | Gel | BCF201 | EN | EN + BCF201 | Optic | Sensodyne |
| 0 | 105 +/− 5 | 105 +/− 4 | 105 +/− 3 | 105 +/− 4 | 105 +/− 3 | 105 +/− 3 |
| 5,000 | 83 +/− 5 | 103 +/− 3 | 96 +/− 9 | 103 +/− 5 | 93 +/− 5 | 103 +/− 4 |
| 10,000 | 73 +/− 7 | 100 +/− 4 | 89 +/− 10 | 102 +/− 3 | 86 +/− 6 | 102 +/− 5 |
| 15,000 | 68 +/− 5 | 100 +/− 4 | 85 +/− 10 | 101 +/− 4 | 80 +/− 4 | 102 +/− 3 |
| 20,000 | 60 +/− 8 | 100 +/− 3 | 79 +/− 11 | 99 +/− 4 | 76 +/− 4 | 100 +/− 4 |

TABLE 6
Roughness of a resin composite surface after 20,000 brushing cycles

| Brushing | | Gel + | Colgate | Colgate | | |
|---|---|---|---|---|---|---|
| cycles | Gel | BCF201 | EN | EN + BCF201 | Colgate Optic | Sensodyne |
| 0 | 7 +/− 2^{a,b} | 7 +/− 3^{a,b} | 8 +/− 2^{a,b} | 9 +/− 4^{a} | 9 +/− 3^{a} | 9 +/− 3^{a} |
| 20,000 | 4 +/− 1^{b} | 6 +/− 2^{a,b} | 42 +/− 9 | 30 +/− 9 | 35 +/− 13 | 65 +/− 22 |

TABLE 7
Roughness of an enamel surface after 20,000 brushing cycles

| Brushing | | Gel + | Colgate | Colgate | Colgate | |
|---|---|---|---|---|---|---|
| cycles | Gel | BCF201 | EN | EN + BCF201 | Optic | Sensodyne |
| 0 | 5 +/− 2^{c} | 5 +/− 1^{c} | 6 +/− 2^{c} | 10 +/− 10^{c,d} | 5 +/− 1^{c} | 6 +/− 3^{c} |
| 20,000 | 5 +/− 2^{c} | 5 +/− 1^{c} | 20 +/− 12^{c} | 9 +/− 5^{d} | 19 +/− 5^{e} | 8 +/− 3^{c,d} |

Additional exemplary glass compositions according to the present disclosure are shown in Table 8, along with glass compositions that are not examples of the present disclosure, which shows the mole percentages of different components.

TABLE 8

| Glass | | | | | | | | | | |
|---|---|---|---|---|---|---|---|---|---|---|
| Identifier | B₂O₃ | Li₂O | RbO₂ | Na₂O | SrO | ZnO | CaF₂ | NaF | KF | SnF₂ |
| BCF301 | 50.0 | 23.2 | 0.0 | 0.0 | 0.0 | 0.0 | 26.8 | 0.0 | 0.0 | 0.0 |
| BCF302 | 50.0 | 0.0 | 0.0 | 0.0 | 0.0 | 0.0 | 50.0 | 0.0 | 0.0 | 0.0 |
| BCF303 | 50.0 | 0.0 | 0.0 | 0.0 | 0.0 | 0.0 | 0.0 | 0.0 | 24.4 | 25.6 |
| BCF304 | 50.0 | 0.0 | 0.0 | 0.0 | 0.0 | 0.0 | 0.0 | 24.2 | 0.0 | 25.8 |
| BCF305 | 50.0 | 23.8 | 0.0 | 0.0 | 0.0 | 0.0 | 0.0 | 0.0 | 0.0 | 26.2 |
| BCF306 | 50.0 | 0.0 | 0.0 | 0.0 | 24.5 | 0.0 | 0.0 | 0.0 | 0.0 | 25.5 |
| BCF307 | 50.0 | 0.0 | 0.0 | 0.0 | 0.0 | 0.0 | 25.1 | 0.0 | 24.9 | 0.0 |
| BCF308 | 50.0 | 0.0 | 0.0 | 0.0 | 0.0 | 24.3 | 0.0 | 0.0 | 25.7 | 0.0 |
| BCF309 | 50.0 | 0.0 | 0.0 | 0.0 | 0.0 | 0.0 | 0.0 | 0.0 | 0.0 | 50.0 |
| BCF310 | 71.2 | 0.0 | 0.0 | 0.0 | 0.0 | 0.0 | 0.0 | 0.0 | 28.8 | 0.0 |
| BCF311 | 50.0 | 0.0 | 0.0 | 24.2 | 0.0 | 0.0 | 0.0 | 0.0 | 0.0 | 25.8 |
| BCF312 | 50.0 | 25.4 | 0.0 | 0.0 | 0.0 | 0.0 | 0.0 | 0.0 | 24.6 | 0.0 |
| BCF313 | 50.0 | 25.0 | 0.0 | 0.0 | 25.0 | 0.0 | 0.0 | 0.0 | 0.0 | 0.0 |
| BCF314 | 50.0 | 24.2 | 25.8 | 0.0 | 0.0 | 0.0 | 0.0 | 0.0 | 0.0 | 0.0 |
| BCF315 | 50.0 | 0.0 | 0.0 | 0.0 | 0.0 | 0.0 | 0.0 | 50.0 | 0.0 | 0.0 |
| BCF316 | 74.2 | 0.0 | 0.0 | 0.0 | 0.0 | 0.0 | 0.0 | 25.8 | 0.0 | 0.0 |
| BCF317 | 50.0 | 0.0 | 0.0 | 0.0 | 0.0 | 0.0 | 25.7 | 24.3 | 0.0 | 0.0 |
| BCF318 | 50.0 | 0.0 | 24.9 | 0.0 | 0.0 | 25.1 | 0.0 | 0.0 | 0.0 | 0.0 |
| BCF319 | 50.0 | 0.0 | 0.0 | 0.0 | 25.2 | 0.0 | 24.8 | 0.0 | 0.0 | 0.0 |
| BCF320 | 50.0 | 25.2 | 0.0 | 0.0 | 0.0 | 0.0 | 0.0 | 24.8 | 0.0 | 0.0 |
| BCF321 | 75.4 | 0.0 | 0.0 | 0.0 | 0.0 | 0.0 | 0.0 | 0.0 | 0.0 | 24.6 |
| BCF322 | 50.0 | 0.0 | 0.0 | 0.0 | 0.0 | 20.0 | 30.0 | 0.0 | 0.0 | 0.0 |
| BCF323 | 50.0 | 0.0 | 0.0 | 23.2 | 0.0 | 0.0 | 0.0 | 0.0 | 26.8 | 0.0 |
| BCF324 | 52.0 | 29.0 | 5.0 | 2.4 | 4.2 | 6.4 | 0.4 | 0.0 | 0.3 | 0.3 |
| BCF325 | 50.0 | 0.0 | 0.0 | 0.0 | 0.0 | 0.0 | 0.0 | 0.0 | 50.0 | 0.0 |
| BCF326 | 50.0 | 0.0 | 24.8 | 0.0 | 0.0 | 0.0 | 25.2 | 0.0 | 0.0 | 0.0 |
| BCF327 | 50.0 | 0.0 | 0.0 | 0.0 | 11.5 | 13.7 | 12.8 | 0.0 | 11.9 | 0.0 |
| BCF328 | 77.4 | 0.0 | 0.0 | 0.0 | 0.0 | 22.6 | 0.0 | 0.0 | 0.0 | 0.0 |
| BCF329 | 50.0 | 0.0 | 0.0 | 0.0 | 0.0 | 0.0 | 0.0 | 26.2 | 23.8 | 0.0 |
| BCF330 | 50.0 | 0.0 | 23.5 | 0.0 | 0.0 | 0.0 | 0.0 | 0.0 | 0.0 | 26.5 |
| BCF331 | 50.0 | 0.0 | 0.0 | 0.0 | 25.6 | 0.0 | 0.0 | 24.4 | 0.0 | 0.0 |
| BCF332 | 50.0 | 0.0 | 25.7 | 0.0 | 0.0 | 0.0 | 0.0 | 24.3 | 0.0 | 0.0 |
| BCF333 | 50.0 | 0.0 | 25.7 | 0.0 | 0.0 | 0.0 | 0.0 | 0.0 | 24.3 | 0.0 |
| BCF334 | 50.0 | 0.0 | 0.0 | 24.8 | 0.0 | 25.2 | 0.0 | 0.0 | 0.0 | 0.0 |
| BCF335 | 70.5 | 0.0 | 0.0 | 29.5 | 0.0 | 0.0 | 0.0 | 0.0 | 0.0 | 0.0 |
| BCF336 | 50.0 | 0.0 | 26.2 | 23.8 | 0.0 | 0.0 | 0.0 | 0.0 | 0.0 | 0.0 |
| BCF337 | 50.0 | 0.0 | 0.0 | 0.0 | 0.0 | 25.9 | 0.0 | 24.1 | 0.0 | 0.0 |
| BCF338 | 73.3 | 26.7 | 0.0 | 0.0 | 0.0 | 0.0 | 0.0 | 0.0 | 0.0 | 0.0 |
| BCF339 | 58.4 | 0.0 | 1.7 | 9.2 | 8.8 | 8.6 | 0.0 | 0.0 | 0.0 | 13.3 |
| BCF340 | 50.0 | 26.4 | 0.0 | 23.6 | 0.0 | 0.0 | 0.0 | 0.0 | 0.0 | 0.0 |
| BCF341 | 50.0 | 0.0 | 0.0 | 0.0 | 0.0 | 0.0 | 25.3 | 0.0 | 0.0 | 24.7 |
| BCF342 | 50.0 | 0.0 | 0.0 | 0.0 | 24.8 | 0.0 | 0.0 | 0.0 | 25.2 | 0.0 |
| BCF343 | 59.5 | 13.4 | 0.0 | 0.0 | 0.0 | 0.0 | 8.7 | 0.0 | 9.2 | 9.2 |
| BCF344 | 50.0 | 0.0 | 0.0 | 0.0 | 0.0 | 25.1 | 0.0 | 0.0 | 0.0 | 24.9 |
| BCF345 | 50.0 | 22.6 | 0.0 | 0.0 | 0.0 | 27.4 | 0.0 | 0.0 | 0.0 | 0.0 |
| BCF346 | 50.0 | 0.0 | 0.0 | 0.0 | 24.9 | 25.1 | 0.0 | 0.0 | 0.0 | 0.0 |
| BCF347 | 50.0 | 0.0 | 0.0 | 25.8 | 24.2 | 0.0 | 0.0 | 0.0 | 0.0 | 0.0 |
| BCF348 | 73.9 | 0.0 | 0.0 | 0.0 | 0.0 | 0.0 | 26.1 | 0.0 | 0.0 | 0.0 |
| BCF349 | 50.0 | 0.0 | 24.9 | 0.0 | 25.1 | 0.0 | 0.0 | 0.0 | 0.0 | 0.0 |
| BCF350 | 50.0 | 0.0 | 0.0 | 27.6 | 0.0 | 0.0 | 22.4 | 0.0 | 0.0 | 0.0 |
| BCF351 | 73.9 | 0.0 | 26.1 | 0.0 | 0.0 | 0.0 | 0.0 | 0.0 | 0.0 | 0.0 |
| BCF352 | 73.5 | 0.0 | 0.0 | 0.0 | 26.5 | 0.0 | 0.0 | 0.0 | 0.0 | 0.0 |
| BCF353 | 50.0 | 11.0 | 0.0 | 13.1 | 0.0 | 11.6 | 10.5 | 3.8 | 0.0 | 0.0 |
| BCF354 | 50.0 | 0.0 | 0.0 | 24.9 | 0.0 | 0.0 | 0.0 | 25.1 | 0.0 | 0.0 |
| BCF357 | 50.0 | 0.0 | 0.0 | 0.0 | 24.9 | 25.1 | 0.0 | 0.0 | 0.0 | 0.0 |
| BCF362 | 73.9 | 0.0 | 26.1 | 0.0 | 0.0 | 0.0 | 0.0 | 0.0 | 0.0 | 0.0 |
| BCF363 | 73.5 | 0.0 | 0.0 | 0.0 | 26.5 | 0.0 | 0.0 | 0.0 | 0.0 | 0.0 |
| BCF364 | 50.0 | 11.0 | 0.0 | 13.1 | 0.0 | 11.6 | 10.5 | 3.8 | 0.0 | 0.0 |

The exemplary glass compositions, and additional compositions, shown in Table 8 were selected based on a design of mixtures (Design Expert 8.0.4, Stat-Ease Inc.) to evaluate the effect of various ranges of components on the glass composition.

The glass compositions were synthesized as described above. Briefly, sufficient amounts of analytical grade reagents (Sigma Aldrich, Canada) needed to form each of the above compositions were weighed. The individual formulations were mixed for 60 mins to ensure homogeneity. Each precursor blend was placed and packed in 50 mL platinum crucibles (Johnson Matthey, Noble Metals, Pennsylvania). The pack crucible was then placed in a furnace (Carbolite, RHF 1600) at room temperature. The furnace was heated (25° C./minute) to an initial dwelling temperature of 600° C. and held for 60 minutes. The temperature was then ramped (20° C./minute) to a final dwelling temperature of 1,100° C. and held for 60 minutes. On removal, each glass melt was quenched between two stainless steel plates.

The following compositions are specific examples of compositions that formed glasses under the above-described quench conditions, such conditions represent one option for standard quench conditions that would be suitable for a production-scale process.

TABLE 9
Examples of compositions that formed
glasses under described quench conditions

| Glass | | | | | | | | | | |
|---|---|---|---|---|---|---|---|---|---|---|
| Identifier | B₂O₃ | Li₂O | RbO₂ | Na₂O | SrO | ZnO | CaF₂ | NaF | KF | SnF₂ |
| BCF301 | 50 | 23.2 | 0 | 0 | 0 | 0 | 26.8 | 0 | 0 | 0 |
| BCF308 | 50 | 0 | 0 | 0 | 0 | 24.3 | 0 | 0 | 25.7 | 0 |
| BCF313 | 50 | 25.4 | 0 | 0 | 0 | 0 | 0 | 0 | 24.6 | 0 |
| BCF315 | 50 | 24.2 | 25.8 | 0 | 0 | 0 | 0 | 0 | 0 | 0 |
| BCF320 | 50 | 0 | 24.9 | 0 | 0 | 25.1 | 0 | 0 | 0 | 0 |
| BCF321 | 50 | 0 | 0 | 0 | 25.2 | 0 | 24.8 | 0 | 0 | 0 |
| BCF322 | 50 | 25.2 | 0 | 0 | 0 | 0 | 0 | 24.8 | 0 | 0 |
| BCF324 | 50 | 0 | 0 | 0 | 0 | 20.0 | 30.0 | 0 | 0 | 0 |
| BCF342 | 50 | 0 | 0 | 0 | 0 | 25.9 | 0 | 24.1 | 0 | 0 |
| BCF357 | 50 | 0 | 0 | 0 | 24.9 | 25.1 | 0 | 0 | 0 | 0 |
| BCF364 | 50 | 11.0 | 0 | 13.1 | 0 | 11.6 | 10.5 | 3.8 | 0 | 0 |

The resulting quenched glasses for the exemplary compositions listed in Table 9 had the following bulk properties:

TABLE 10
Bulk properties for some exemplary glasses

| | | | Glass Transition Temp | | |
|---|---|---|---|---|---|
| Glass | Density | % | (° C.) | | |
| Identifier | (g/cm³) | Crystallinity | Onset | Inflection | Fictive |
| BCF301 | 2.5879 (±0.03) | 1.20 | 425.8 | 450.7 | 451 |
| BCF308 | 2.7420 (±0.004) | 0.9 | 415.5 | 435.7 | 426 |
| BCF313 | 2.3125 (±0.01) | 1.3 | 379.1 | 392.2 | 387 |
| BCF315 | 2.8084 (±0.07) | 0.7 | 310.3 | 310.6 | 294 |
| BCF320 | 3.1301 (±0.04) | 0.4 | 410.0 | 410.2 | 436 |
| BCF321 | 3.2655 (±0.009) | 0.9 | 512.6 | 526.0 | 521 |
| BCF322 | 2.3414 (±0.008) | 0.5 | 387.2 | 398.2 | 352 |
| BCF324 | 3.1335 (±0.009) | 2.1 | 530.4 | 519.3 | 546 |
| BCF342 | 2.9056 (±0.01) | 1.8 | 462.3 | 474.6 | 482 |
| BCF357 | 3.5263 (±0.004) | 0.5 | 552.2 | 578.2 | 605 |
| BCF364 | 2.6382 (±0.02) | 1 | 391.6 | 401.8 | 402 |

The resulting quenched glasses for the exemplary compositions listed in Table 9 were ground/milled separately within a planetary micro mill (Pulverisette 7, Fritsch, Germany) and sieved with ASTM E-11 compliant sieves (Cole Palmer, U.S.A) to obtain particles of <25 μm.

The particle size distribution for the exemplary glasses listed in Table 9 is shown in Table 11. BCF315, BCF326 degraded in the deionized water too quickly to obtain accurate particle size measurements.

TABLE 11
Particle size distribution (μm)

| Glass | Dx | Dx | Dx |
|---|---|---|---|
| Identifier | (10) | (50) | (90) |
| BCF301 | 3.15 | 11.00 | 24.30 |
| BCF308 | 3.07 | 9.71 | 22.10 |
| BCF313 | 4.70 | 11.00 | 21.00 |
| BCF315 | * | * | * |
| BCF320 | 3.11 | 9.23 | 20.60 |
| BCF321 | 2.94 | 9.73 | 20.70 |
| BCF322 | 3.96 | 10.40 | 21.10 |
| BCF324 | 3.63 | 9.39 | 19.80 |
| BCF342 | 3.36 | 10.90 | 24.30 |
| BCF357 | 2.79 | 9.66 | 21.90 |
| BCF364 | 2.9 | 8.73 | 19.8 |

The particles of the exemplary glasses listed in Table 9 were evaluated for mass loss and fluoride release in a buffered saline solution at 1, 4 and 24 hours. Samples were prepared in 15 ml conical test tubes (n=3), which were weighed and recorded. 0.1 grams of each glass powder (<25 microns) were separately weighed out and placed in 10 mL TRIS buffered saline (BioUltra, Sigma Aldrich, Canada) in the weighed 15 mL Falcon tubes. The tubes were sealed with parafilm before being placed in a shaking incubator at 37° C. and agitated at 120 rpm for four separate time points: 5 mins, 30 mins, 1 hr, 3 hrs, 24 hours and 48 hours. After the specified time points elapsed, the tubes were removed from the incubator and the solutions were immediately centrifuged (Eppendorf, Centrifuge 5702) at 3.0 RCF/4.4 RPM for 15 minutes. The supernatant was decanted into fresh 15 mL Falcon tubes. Additionally, samples of the 48-hour incubated powders were re-suspended in 10 mL of fresh TRIS buffered saline by vortex mixing, and allowed to incubate for an additional 8 hours (for a total of 56 hours of incubation). The re-incubated powders were processed in an identical manner as the other samples. The pellets were dried in an oven at 50° C. in their respective Falcon tubes.

The release of fluoride ions was measured using an Accumet AB250 pH/ion selective meter equipped with an Fluoride electrode (Fisher Scientific). To calibrate the probe, 6 standard solutions were prepared using a fluoride analytical standard specifically for ion selective electrodes (NaF, 0.1 F, Sigma Aldrich, Canada). The fluoride concentrations of the standards were synthesized as follows: 1000 ppm, 100 ppm, 10 ppm, 1 ppm, 0.1 ppm and 0.01 respectively, using TRIS buffered saline (BioUltra, Sigma Aldrich, Canada) as the solvent. TISAB concentrate (4.5 mL) was added to each standard before calibration (as per manufacturer's instructions). Once the probe was calibrated, the slope of the standard was checked to ensure it was within range from the instructions of use. TISAB concentrate (1.0 mL) was added to the decanted supernatants and were then measured for its fluoride concentrations using the calibrated probe. The ion concentrations are reported as the average±SD.

The mass losses and ppm values of released fluoride are shown in Table 12. BCF314 degraded completely in the buffered saline solution before the 1 hour time point, and additionally did not include any source of fluoride.

TABLE 12
Mass loss and fluoride release for
exemplary glasses at 1, 4 and 24 hours

| | Mass Loss | | | Fluoride Release | | |
|---|---|---|---|---|---|---|
| | (%) | | | (ppm) | | |
| Glass Identifier | 1 | 4 | 24 | 1 | 4 | 24 |
| BCF301 | 45.23 | 36 | 62 | 22 | 26 | 24 |
| BCF308 | 13.74 | 13 | 14 | 50 | 54 | 67 |
| BCF313 | 100% | N/A | N/A | 270 | N/A | N/A |
| BCF315 | N/A | N/A | N/A | N/A | N/A | N/A |
| BCF320 | 56.43 | 63 | 58 | 1 | 1.2 | 1 |
| BCF321 | 38.55 | 59 | 65 | 21 | 21 | 18 |
| BCF322 | 100% | N/A | N/A | 329 | N/A | N/A |
| BCF324 | 7.554 | 20 | 13 | 15 | 11 | 6.7 |
| BCF342 | 12.37 | 8.1 | 18 | 22 | 22 | 29 |
| BCF357 | 14.66 | 13 | 18 | 0 | 0.1 | 0 |
| BCF364 | 13.488 | 21 | 12 | 30 | 32 | 31 |

The compositions listed in Table 8 reflect a design space. The results of the tested compositions provided the following equations, which may allow for the relative comparison of different compositions and/or which may be useful to identify trends associated with different components of the compositions. While experimental and modeling error prevents absolute prediction of glass properties, the equations may be used to guide and refine glass composition design. When used together, these models may help suggest which factors may be traded off in the tailoring of multi-component compositions within the tested composition space. In the following equations, the values for the listed components are in percentages (not fractions or decimals). For example, 50 mol % of B₂O₃ would be “50” (and not “0.5”).

Glass is generally expected to form under the tested quench conditions if the following formula is less than or equal to 1.60:

(2.01*e^{y}+0.99)/(1+e^{y})

where y=−0.086622*[B₂O₃]+0.14169*[Li₂O]−0.565849*[ZnO]+0.192175*[Na₂O]−0.461537*[CaF₂]+0.036636*[KF]+0.00365*[NaF]+0.191201*[SnF₂]+0.192612*[RbO₂]+0.199999*[SrO]+0.01393*[B₂O₃]*[ZnO]+0.012239*[B₂O₃]*[CaF₂]−0.012412*[Li₂O]*[CaF₂]−0.013904*[Li₂O]*[RbO₂]−0.010857*[ZnO]*[CaF₂]−0.013296*[ZnO]*[RbO₂]−0.010699*[ZnO]*[SrO]+0.010128*[CaF₂]*[KF]−0.012103*[CaF₂]*[SrO].

The density of a glass may be generally predicted using the following formula: ρ=0.018783*[B₂O₃]+0.026444*[Li₂O]+0.046191*[ZnO]+0.033814*[Na₂O]+0.039196*[CaF₂]+0.026997*[KF]+0.029458*[NaF]+0.049441*[SnF₂]+0.047057*[R_{b}O₂]+0.054984*[SrO]. Glass densities from about 1.3 g/cm³ to about 2.2 g/cm³ may particularly useful in non-aqueous oral care formulations. Glycerol and silica, which are the primary liquid and solid components of a non-aqueous toothpaste, have densities of 1.3 and 2.2 g/cm³, respectively.

The glass transition temperature (T_{g}) may be generally predicted using the following formula: T_{g}=3.49398*[B₂O₃]+3.66342*[Li₂O]+6.38755*[ZnO]+6.23689*[Na₂O]+6.43079*[CaF₂]+3.31695*[KF]+5.04074*[NaF]+9.88761*[SnF₂]+3.29777*[RbO₂]+10.51264*[SrO]. It should be understood that phase separated glasses may present multiple glass transitions, the magnitude of which is not necessarily representative of the volume distribution of the phases. While the above equation predicts the onset of a glass transition, the predicted onset may not be the predominant glass transition of the composition if phase separation occurs. Accordingly, a predicted glass transition temperature may be significantly different from the measured predominant glass transition temperature.

The equation related to percent of mass loss after 1 hour under the tested conditions is:

(100*e^{y})/(1+e^{y})

where y=0.088098*[B₂O₃]+0.062481*[Li₂O]−0.262486*[ZnO]+0.055442*[Na₂O]−0.165517*[CaF₂]+0.089171*[KF]+0.075875*[NaF]+0.10439*[SnF₂]+0.109897*[RbO₂]−0.089987*[SrO]. The above equation is highly predictive for identifying glass compositions which demonstrate complete dissolution within 1 hour under the tested conditions, and may be useful for identifying other glasses which degrade under this time frame. Further, although the equation does not provide accurate mass loss estimates for slower degrading compositions, the equation may be useful to predict the relative changes in degradation which could be expected to occur with changes of the composition. Such relative changes may be used as a guide in glass composition design.

The equation related to release of fluoride (in ppm) after 1 hour under the tested conditions is:

(2750*e^{y})/(1+e^{y})

where y=−0.05785*[B₂O₃]−0.158337*[Li₂O]−0.170872*[ZnO]−0.184773*[Na₂O]+0.05638*[CaF₂]+0.101381*[KF]+0.053886*[NaF]−0.307462*[SnF₂]−0.183034*[RbO₂]−0.184126*[SrO]. Although the above equation does not provide an accurate estimate of the amount of fluoride released for all glass compositions, the model may still be useful to predict the relative changes in fluoride release which could be expected to occur with changes of the composition.

PBF1 was synthesized by: weighing 11.60 g of B₂O₃, 5.30 g of Na₂CO₃, 2.69 g of MgO, 3.33 g of CaCO₃, and 0.7 g of CaF₂ (Sigma Aldrich, Canada). The starting materials were mixed for 60 mins to ensure homogeneity. The blend was placed and packed in 50 mL platinum crucibles (Johnson Matthey, Noble Metals, Pennsylvania). The pack crucible was then placed in a furnace (Carbolite, RHF 1600) at room temperature. The furnace was heated (25° C./minute) to an initial dwelling temperature of 600° C. and held for 60 minutes. The temperature was then ramped (20° C./minute) to a final dwelling temperature of 1,200° C. and held for 60 minutes. On removal, the glass melt was quenched between two stainless steel plates. The resulting quenched glasses were ground/milled separately within a planetary micro mill (Pulverisette 7, Fritsch, Germany) and sieved with ASTM E-11 compliant sieves (Cole Palmer, U.S.A) to obtain particles of <25 μm.

Comparative glass compositions (referred to as Comparative Examples (CE) 1 and 2) were synthesized similarly, using: 5.80 g B₂O₃, 23.66 g P₂O₅, 5.30 g Na₂CO₃, 1.34 g MgO, 6.67 g CaCO₃, and 0.70 g CaF₂ to result in: CE1 with about 25 mol % B₂O₃, about 25 mol % P₂O₅, about 15 mol % Na₂O, about 10 mol % MgO, about 20 mol % CaO, and about 5 mol % CaF₂; and 5.80 g B₂O₃, 23.66 g P₂O₅, 7.07 Na₂CO₃, 1.34 g MgO, 5.00 g CaCO₃, and 0.70 g CaF₂ to result in CE2 with about 25 mol % B₂O₃, about 25 mol % P₂O₅, about 20 mol % Na₂O, about 10 mol % MgO, about 15 mol % CaO, and about 5 mol % CaF₂.

The density of the glass powders were measured using an AccuPyc 1340 helium pycnometer (Micromeritics, USA) equipped with a 1 cm³ insert. Prior to use, a standard with a volume of 0.718512 cm³ was used to calibrate the pycnometer. For glass analysis, the insert was packed with about 0.5 to 0.7 grams of glass powder. Three samples of each of the glasses were run and each measurement is the mean of 10 readings.

The density of PBF1 was measured as 2.5951±0.0072 g/cm³. The density of CE1 was measured as 2.7079±0.0021 g/cm³. The density of CE2 was measured as 2.6749±0.0013 g/cm³.

The release of fluoride and the loss of mass was measured for PBF1, CE1 and CE2. Samples were prepared in 15 ml conical test tubes (n=3), which were weighed and recorded. 0.1 grams of each glass powder (<25 microns) were separately weighed out and placed in 10 mL TRIS buffered saline (BioUltra, Sigma Aldrich, Canada) in the weighed 15 mL Falcon tubes. The tubes were sealed with parafilm before being placed in a shaking incubator at 37° C. and agitated at 120 rpm for four separate time points: 5 mins, 30 mins, 1 hr, 3 hrs, 24 hours and 48 hours. After the specified time points elapsed, the tubes were removed from the incubator and the solutions were immediately centrifuged (Eppendorf, Centrifuge 5702) at 3.0 RCF/4.4 RPM for 15 minutes. The supernatant was decanted into fresh 15 mL Falcon tubes. Additionally, samples of the 48-hour incubated powders were re-suspended in 10 mL of fresh TRIS buffered saline by vortex mixing, and allowed to incubate for an additional 8 hours (for a total of 56 hours of incubation). The re-incubated powders were processed in an identical manner as the other samples. The pellets were dried in an oven at 50° C. in their respective Falcon tubes.

The release of fluoride ions was measured using an Accumet AB250 pH/ion selective meter equipped with an Fluoride electrode (Fisher Scientific). To calibrate the probe, 6 standard solutions were prepared using a fluoride analytical standard specifically for ion selective electrodes (NaF, 0.1 F, Sigma Aldrich, Canada). The sodium fluoride concentrations of the standards were synthesized as follows: 1000 ppm, 100 ppm, 10 ppm, 1 ppm, 0.01 ppm and 0.001 respectively, using TRIS buffered saline (BioUltra, Sigma Aldrich, Canada) as the solvent. TISAB concentrate (4.5 mL) was added to each standard before calibration (as per manufacturer's instructions). Once the probe was calibrated, the slope of the standard was checked to ensure it was within range from the instructions of use. TISAB concentrate (1.0 mL) was added to the decanted supernatants and were then measured for its fluoride concentrations using the calibrated probe. The ion concentrations are reported as the average±SD.

The amount of fluoride ion released by PBF1 was measured as: 89±2 ppm at 5 mins; 94±3 ppm at 30 mins; 105±5 ppm at 1 hour; and 94±7 ppm at 3 hours. There was no measurable fluoride ion released by CE1 or CE2.

The loss of mass was calculated by comparing the mass of the dried samples after their exposure to the TRIS buffered saline to the initial mass of the samples. The mass loss for PBF1 was: 42.0±2.1% after 5 minutes; 47.3±2.7% after 30 minutes; 51.5±4.3% after 1 hour; 41.7±5.7% after 3 hours; 70.1±6.8% after 24 hours; and 100% after 48 hours.

The particle size of seven different samples of PBF1 was measured using a Malvern Mastersizer 3000 model laser diffraction particle size analyzer. Glass particles were separately suspended in distilled water to obtain an obscuration value between 2-5%. Prior to analysis, the glass powder was stored in a vacuum desiccator and was removed for analysis for 3×5 runs lasting approximately 20 secs/run. Suspensions were measured using both a blue (λ=470 nm) and red (λ=632.8 nm) laser (n=5).

TABLE 13
Particle size distribution for PBF1

| | Dx10 | Dx50 | Dx90 |
|---|---|---|---|
| | (μm) | (μm) | (μm) |
| PBF1.1 | 3.18 | 11.9 | 26.9 |
| PBF1.2 | 3.44 | 10.5 | 22.2 |
| PBF1.3 | 3.26 | 9.99 | 22.1 |
| PBF1.4 | N/A | N/A | N/A |
| PBF1.5 | 3.35 | 12.3 | 27.4 |
| PBF1.6 | 3.44 | 10.3 | 22.3 |
| PBF1.7 | 4.84 | 13.2 | 27.1 |

Apatite formation in simulated body fluid was confirmed for PBF1, but was not evident with CE1 or CE2. Simulated Body Fluid was synthesized as per the methods and instructions published by Kokubo and Takadama (Kokubo, T. and Takadama, H. Biomaterials (2006) 27:15, U.S. Plant Pat. No. 2,907-2915).

1 L batches of SBF were prepared in 1000 mL Nalgene bottle (FEP bottle). The prepared SBF was stored at room temperature for 24 hrs immediately after synthesis to ensure stability before experimental use. The SBF was preserved in a Nalgene bottle with the lid on tightly and kept at 6° C. if not needed immediately (for up to 30 days for experimental use).

As per the TCO4 method (published in Maçon, A. L. B., Kim, T. B., Valliant, E. M. et al. J Mater Sci: Mater Med (2015) 26:115) 0.75 g of glass powder of each glass composition (n=3) was immersed in 50 mL of SBF, as synthesized per above, in polyethylene containers. Containers were then placed in an incubating orbital shaker at 37° C. and agitated at 120 rpm for 3 time points: 30 mins, 3 hrs and 12 hrs. After the time points elapsed, each specimen was vacuum filtered with Whatman 42 or 5 grade filter paper (particle retention of 2.5 μm) to collect the solid material from the solution. The solids were immediately washed with distilled water and acetone to stop any further reaction.

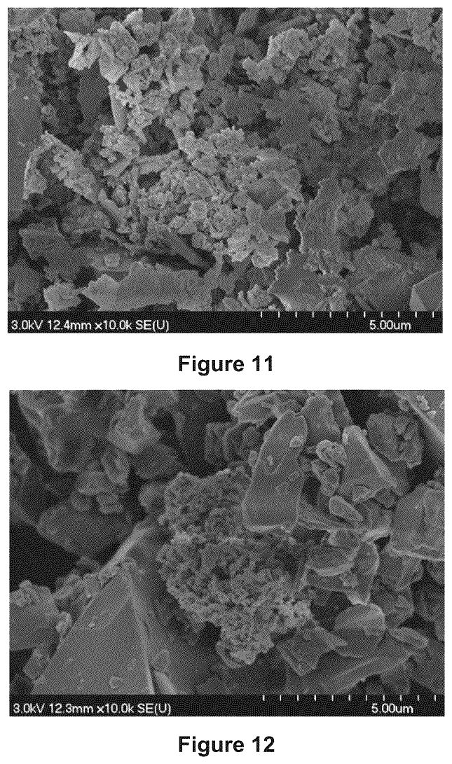

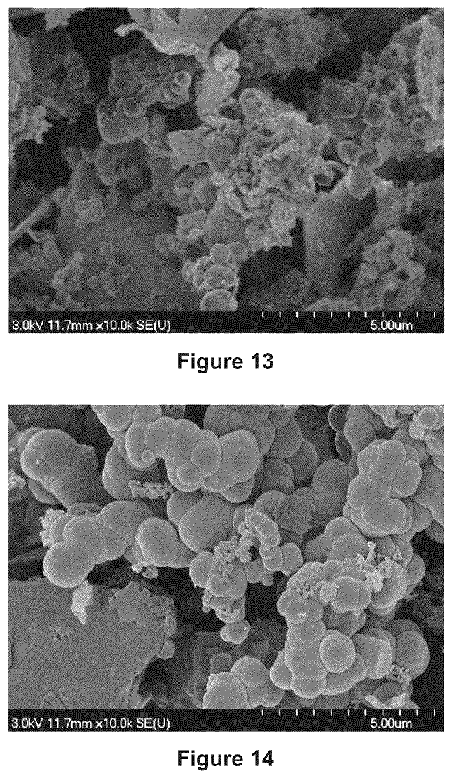

The filtered specimens were dried in a vacuum desiccator for further analysis. Imaging of each specimen was performed using a Hitachi S-4700 FEG (Hitachi, Chula Vista, Ca) scanning electron microscope operating at 3 KV and 15 mA under magnification of 1000× and 10000×. Samples were mounted on stubs using double sided carbon tape and sputter coated with gold-palladium for 70 s (Leica EM ACE200, Wetzlar, Germany). The scanning electron microscope images of PBF1 at 30 minutes, 3 hours and 12 hours are shown in FIGS. 11 to 14.

PBF1 was also assessed for dentin tubule occlusion by developing an application protocol and statistical analysis of SEM images graded by two assessors according to a categorical occlusion scale. Sections of human dentin (about 1 to 1.5 mm thick) were prepared from the crowns of caries-free, unrestored molars, perpendicular to the long axis of the roots, using a diamond disc saw. Each section was etched for 2 minutes with 10% citric acid, followed by water rinsing for 60 seconds, sonication for 2 minutes and a further water rinse for 60 seconds. Each section was placed into a 25 mm diameter mould and covered with 3 mm-deep acrylic resin. Once the resin hardened, the dentin face was polished sequentially with 800, and 2500-grit paper to a mirror finish. Following a deionised water rinse, the surface was etched, sonicated and rinsed once more. Sample integrity, tubule density and patency were once again checked under a light microscope, and then with SEM.

A single dentin sample was assigned to each treatment group. The dentin sample was treated with (i) an unformulated mixture of exemplary glass particles, (ii) a test toothpaste that included a mixture of exemplary glass particles, or (iii) a control toothpaste without any additional glass particles. The unformulated mixture was applied using a non-powder nitrile gloved finger for 10 seconds. The test and control toothpastes were applied to the sample with an electric toothbrush for 10 second. The toothpaste was left for 30 seconds before being rinsed until all visible paste was removed. This was repeated for a total of 4 applications of the toothpaste.

Dentin samples were dried in an oven for 1 hour at 37° C., sputter coated with gold, and visualized using a Phenom ProX Scanning Electron Microscope. Five images at ×3000 magnification were taken of different portions of each sample, in which the tubules were perpendicular to the surface. Each ×3000 micrograph was examined by two single-blinded assessors for the extend of dentin tubule occlusion based on a five-point categorical scale. The grading classification was defined as:
1. Occluded (100% occlusion)
2. Mostly occluded (75% occlusion)
3. Equal (50% occlusion)
4. Mostly unoccluded (25% occlusion)
5. Unoccluded (0% occlusion)

Mean scores for each image were derived from the scores of the two assessors. Standard deviations were calculated, though no formal statistical comparisons were made due to the fact that only one dentin sample was used per treatment group.

Seven different treatment groups were tested, as outlined in Table 14.

TABLE 14
Treatment groups for test of dentin tubule occlusion

| Treatment | | |
|---|---|---|
| Group | Test Article | Materials |
| 1 | No treatment of | N/A |
| | dentin sample | |
| 2 | Test Article #1 | 0.1 g PBF1 |
| 3 | Test Article #2 | 0.0125 g PBF1 + 0.25 g Sensodyne ™ |
| | | Complete Protection (5% w/w) |
| 4 | Test Article #3 | 0.0375 g PBF1 + 0.25 g Sensodyne ™ |
| | | Complete Protection (15% w/w) |
| 5 | Control Article #1 | 0.25 g Sensodyne ™ Complete |
| | | Protection |
| 6 | Control Article #2 | 0.25 g Colgate ™ Pro-Relief |
| 7 | Control Article #3 | 0.25 g Sensodyne ™ Repair & Protect |

As discussed above, each sample treatment group was tested on a dentin sample and five SEM micrographs of each sample were taken at ×3000. Each micrograph was categorically assessed by two assessors. The average score for each micrograph, and the five micrographs per sample, were combined to obtain a group mean score and standard deviation (see Table 15).

TABLE 15
Mean occlusion score for
different treatment groups

| Treatment | Group Mean |
|---|---|
| Group | (±SD) |
| 1 | 4.90 (±0.22) |
| 2 | 1.50 (±0.0) |
| 3 | 2.90 (±1.02) |
| 4 | 2.40 (±0.42) |
| 5 | 3.60 (±0.22) |
| 6 | 3.60 (±0.22) |
| 7 | 3.20 (±0.45) |

The mean baseline score of 4.90 for treatment group 1 illustrates that virtually all dentin tubules were un-occluded. The mean score of 1.50 for the unformulated PBF1 rubbed directly into the dentin sample illustrates a nearly complete tubule blockage. The treatment groups 5, 6 and 7 (control groups lacking PBF1 or any other glass composition according to the present disclosure) had mean occlusion scores from 3.2 to 3.6. The treatment groups 3 and 4 (commercial toothpaste formulated with 5% or 15% PBF1 w/w) had lower mean occlusion scores, indicating a greater degree of tubule occlusion. The degree of occlusion for the commercially available toothpaste Sensodyne™ Complete Protection increased from about 30% occlusion (score 3.6) to about 50% occlusion (score 2.5) when 15% w/w/of PBF1 was added.

PBF1 was further assessed for dentin tubule occlusion using a 5% w/w PBF1 sodium lauryl sulfate (SLS) paste. In this assessment, the PBF1-toothpaste and control toothpaste were applied to three different dentin samples for each treatment group. The samples were each brushed once for two minutes with the treatment toothpaste. Specifically, each dentin sample was brushed with 0.25 g of a treatment toothpaste for 120 seconds and subsequently rinsed with DI water for 30 seconds. The 5% PBF1-SLS paste resulted in a mean occlusion score of 2.7±0.84. The SLS paste without the PBF1 resulted in a mean occlusion score of 3.80±1.03. A control test using Sensodyne™ Repair & Protect resulted in a mean occlusion score of 3.90±0.66.

PBF1-Na was prepared following the protocols discussed above. Briefly, the glass was synthesized by: weighing 11.05 g of B₂O₃, 3.36 g of Na₂CO₃, 2.56 g of MgO, 4.77 g of CaCO₃, and 1.33 g of NaF (Sigma Aldrich, Canada). The starting materials were mixed for 60 mins to ensure homogeneity. The blend was placed and packed in 50 mL platinum crucibles (Johnson Matthey, Noble Metals, Pennsylvania). The pack crucible was then placed in a furnace (Carbolite, RHF 1400) at room temperature. The furnace was heated (25° C./minute) to an initial dwelling temperature of 600° C. and held for 60 minutes. The temperature was then ramped (20° C./minute) to a final dwelling temperature of 1,200° C. and held for 60 minutes. On removal, the glass melt was quenched between two stainless steel plates. The resulting quenched glasses were ground/milled separately within a planetary micro mill (Pulverisette 6, Fritsch, Germany) and sieved with ASTM E-11 compliant sieves (Cole Palmer, U.S.A) to obtain particles of <25 μm.

The particle size of ten different samples of PBF1-Na was measured as discussed above.

TABLE 16
Particle size distribution for PBF1-Na

| | Dx10 | Dx50 | Dx90 |
|---|---|---|---|
| | (μm) | (μm) | (μm) |
| PBF1-Na.1 | 4.7 | 14.9 | 35.2 |
| PBF1-Na.2 | 4.6 | 14.4 | 31.2 |
| PBF1-Na.3 | 4.2 | 13.3 | 29.9 |
| PBF1-Na.4 | 4.5 | 14.1 | 30.7 |
| PBF1-Na.5 | 4.3 | 12.0 | 26.7 |
| PBF1-Na.6 | 4.4 | 13.9 | 30.5 |
| PBF1-Na.7 | 4.1 | 12.1 | 26.2 |
| PBF1-Na.8 | 4.3 | 12.2 | 25.5 |
| PBF1-Na.9 | 4.0 | 11.8 | 25.7 |
| PBF1-Na.10 | 4.2 | 13.3 | 29.7 |
| Average | 4.3 | 13.2 | 29.1 |

The density, % crystallinity, and glass transition temperatures for the ten different samples were also measured as discussed above.

TABLE 17
Bulk properties for PBF1-Na

| | | | Glass Transition Temp | | |
|---|---|---|---|---|---|
| Glass | Density | % | (° C.) | | |
| Identifier | (g/cm³) | Crystallinity | Onset | Inflection | Fictive |
| PBF1-Na.1 | 2.543 (±0.004) | 1.5 | 494.2 | 508.3 | 518.3 |
| PBF1-Na.2 | 2.537 (±0.004) | 1.8 | 492.6 | 506.1 | 519.6 |
| PBF1-Na.3 | 2.546 (±0.003) | 1.8 | 493.7 | 506.6 | 520.6 |
| PBF1-Na.4 | 2.544 (±0.003) | 1.9 | 493.3 | 506.7 | 519.9 |
| PBF1-Na.5 | 2.548 (±0.004) | 1.8 | 492.5 | 505.6 | 520.0 |
| PBF1-Na.6 | 2.544 (±0.004) | 1.8 | 493.0 | 507.0 | 519.3 |
| PBF1-Na.7 | 2.549 (±0.005) | 1.6 | 491.6 | 504.5 | 517.1 |
| PBF1-Na.8 | 2.546 (±0.004) | 1.8 | 491.3 | 506.6 | 520.4 |
| PBF1-Na.9 | 2.545 (±0.004) | 2.0 | 494.1 | 505.3 | 520.4 |
| PBF1-Na.10 | 2.541 (±0.004) | 1.9 | 491.7 | 506.3 | 517.7 |
| Average | 2.544 (±0.005) | 1.8 | 492.8 | 506.3 | 519.3 |

The mass loss and fluoride release after 24 hours for the ten different samples were also measured as discussed above.

TABLE 18
Mass loss and fluoride release
after 24 hours for PBF1-Na

| | Mass | Fluoride |
|---|---|---|
| Glass | Loss | Release |
| Identifier | (%) | (ppm) |
| PBF1-Na.1 | 70.3 | 92.0 |
| PBF1-Na.2 | 71.7 | 91.2 |
| PBF1-Na.3 | 71.7 | 88.9 |
| PBF1-Na.4 | 73.3 | 88.4 |
| PBF1-Na.5 | 73.0 | 87.5 |
| PBF1-Na.6 | 73.3 | 95.0 |
| PBF1-Na.7 | 72.0 | 98.8 |
| PBF1-Na.8 | 72.0 | 93.9 |
| PBF1-Na.9 | 72.7 | 94.2 |
| PBF1-Na.10 | 73.3 | 97.6 |
| Average | 72.3 | 92.8 |

In the preceding description, for purposes of explanation, numerous details are set forth in order to provide a thorough understanding of the examples. However, it will be apparent to one skilled in the art that these specific details are not required. Accordingly, what has been described is merely illustrative of the application of the described examples and numerous modifications and variations are possible in light of the above teachings.

Since the above description provides examples, it will be appreciated that modifications and variations can be effected to the particular examples by those of skill in the art. Accordingly, the scope of the claims should not be limited by the particular examples set forth herein, but should be construed in a manner consistent with the specification as a whole.

What is claimed is:

1. A glass composition comprising:
from about 50 mol % to about 95 mol % of B₂O₃;
from about 5 mol % to about 50 mol % of one or more glass components selected from the group consisting of: Li₂O, Rb₂O, K₂O, Na₂O, SrO, CaO, MgO, and ZnO;
0 mol % of CuO;
less than 0.1 mol % of BaO; and
less than 0.1 mol % of P₂O₅;
wherein the glass composition comprises less than 30 mol % of Rb₂O,
wherein the glass composition is at least a quaternary system,
wherein the glass composition loses at least 5 mass % within 24 hours when exposed to a buffered saline solution, and
wherein the glass composition is a particulate material that comprises particles that are from about 1 to about 50 μm in size.

2. The glass composition according to claim 1, wherein the glass composition comprises from about 5 mol % to about 50 mol % of one or more glass components selected from the group consisting of: Li₂O, Rb₂O, K₂O, Na₂O, SrO, and ZnO; and wherein the glass composition comprises less than 0.1 mol % of CaO and less than 0.1 mol % of MgO, such as substantially no CaO and substantially no MgO.

3. The glass composition according to claim 1, wherein the glass composition comprises from about 5 mol % to about 50 mol % of one or more glass components selected from the group consisting of: Li₂O, Rb₂O, K₂O, SrO, and ZnO; and wherein the glass composition comprises less than 0.1 mol % of CaO, less than 0.1 mol % of MgO, and less than 0.1 mol % of Na₂O, such as substantially no CaO, substantially no MgO, and substantially no Na₂O.

4. The glass composition according to any one of claims 1 to 3, wherein the glass composition comprises about 50 mol % B₂O₃.

5. The glass composition according to claim 1, comprising from about 50 mol % to about 80 mol % of B₂O₃.

6. The glass composition according to claim 1 or 5, comprising from about 5 mol % to about 40 mol % of the glass component selected from the group consisting of: Li₂O, Rb₂O, K₂O, Na₂O, SrO, CaO, MgO, ZnO and any combination thereof.

7. The glass composition according to claim 1, 5 or 6, wherein the glass composition comprises B₂O₃ and Li₂O.

8. The glass composition according to claim 1, 5 or 6, wherein the glass composition comprises B₂O₃, Li₂O and Rb₂O:
from about 5 mol % to about 25 mol % Li₂O, and
from about 5 mol % to about 25 mol % Rb₂O.

9. The glass composition according to claim 8, wherein the glass composition comprises about 70 mol % B₂O₃.

10. The glass composition according to claim 8, wherein the glass composition comprises about 50 mol % B₂O₃.

11. The glass composition according to claim 1, 5 or 6, wherein the glass composition comprises B₂O₃, Li₂O, and ZnO, and optionally Na₂O.

12. The glass composition according to claim 11, wherein the glass composition comprises:
from about 5 mol % to about 30 mol % Li₂O, and
from about 5 mol % to about 30 mol % ZnO,
and optionally from about 5 mol % to about 15 mol % Na₂O.

13. The glass composition according to claim 12, wherein the glass composition comprises about 70 mol % B₂O₃.

14. The glass composition according to claim 12, wherein the glass composition comprises about 50 mol % B₂O₃.

15. The glass composition according to claim 1, 5 or 6, wherein the glass composition comprises B₂O₃, and ZnO.

16. The glass composition according to claim 15, wherein the glass composition comprises from about 5 mol % to about 30 mol % ZnO.

17. The glass composition according to claim 16, wherein the glass composition comprises about 50 mol % B₂O₃.

18. The glass composition according to any one of claims 15 to 17, wherein the glass composition further comprises RbO₂.

19. The glass composition according to claim 18, wherein the glass composition comprises about 5 mol % to about 30 mol % RbO₂.

20. The glass composition according to claim 1, 5 or 6, wherein the glass composition comprises B₂O₃, and SrO.

21. The glass composition according to claim 20, wherein the glass composition comprises from about 5 mol % to about 30 mol % SrO.

22. The glass composition according to claim 21, wherein the glass composition comprises about 50 mol % B₂O₃.

23. The glass composition according to any one of claims 20 to 22, wherein the glass composition further comprises ZnO.

24. The glass composition according to claim 23, wherein the glass composition comprises about 5 mol % to about 30 mol % ZnO.

25. The glass composition according to any one of claims 1 to 24, wherein the glass composition comprises 0 mol % of CuO, 0 mol % of BaO, and 0 mol % of P₂O₅.

26. The glass composition according to claim 1, wherein less than 20 mol %, such as less than 15 mol %, less than 10 mol %, or less than 5 mol % of the glass composition is CaO, MgO, and Na₂O.

27. The glass composition according to any one of claims 1 to 26, wherein at least 75% of the particles are smaller than 50 μm in size.

28. The glass composition according to any one of claims 1 to 26, wherein at least 85% of the particles are smaller than 50 μm in size.

29. The glass composition according to any one of claims 1 to 26, wherein at least 95% of the particles are smaller than 50 μm in size.

30. The glass composition according to any one of claims 1 to 29, wherein at least 5% of the particles are smaller than 7 μm in size.

31. The glass composition according to any one of claims 1 to 29, wherein:
at least 5% of the particles are smaller than 35 μm in size,
at least 5% of the particles are smaller than 15 μm in size, and
at least 5% of the particles are smaller than 7 μm in size.

32. The glass composition according to any one of claims 1 to 30, wherein:
at least 5% of the particles are from about 15 μm to about 35 μm in size,
at least 5% of the particles are from about 6 μm to about 15 μm in size, and
at least 5% of the particles are from about 3 μm to about 7 μm in size.

33. The glass composition according to any one of claims 1 to 26, wherein:
about 10% of the particles are smaller than 5 μm in size,
about 50% of the particles are smaller than 15 μm in size, and
about 90% of the particles are smaller than 30 μm in size.

34. The glass composition according to any one of claims 1 to 33, wherein the glass composition loses at least 20 mass % within 24 hours when exposed to a buffered saline solution.

35. The glass composition according to any one of claims 1 to 33, wherein the glass composition loses at least 40 mass % within 24 hours when exposed to a buffered saline solution.

36. The glass composition according to any one of claims 1 to 33, wherein the glass composition loses at least 60 mass % within 24 hours when exposed to a buffered saline solution.

37. The glass composition according to any one of claims 1 to 33, wherein the glass composition loses at least 80 mass % within 24 hours when exposed to a buffered saline solution.

38. The glass composition according to any one of claims 1 to 37, further comprising a source of fluoride in the form of: CaF₂, NaF, Na₂PO₃F, KF, SnF₂, or any combination thereof.

39. The glass composition according to claim 38, wherein the source of fluoride is about 30 mol % of the glass composition.

40. The glass composition according to claim 38, wherein the source of fluoride is about 15 mol % of the composition.

41. The glass composition according to claim 38, wherein the source of fluoride is from about 1 mol % to about 10 mol % of the composition.

42. The glass composition according to claim 38, wherein the source of fluoride is from about 1 mol % to about 5 mol % of the composition.

43. The glass composition according to any one of claims 38 to 42, wherein the fluoride is present in an amount sufficient that 0.1 g of the particulate material releases the fluoride into 10 ml of a buffered saline solution at an average rate of about 1 ppm/hr to about 15 ppm/hr over 1, 2, 4, 8, 12, 18 or 24 hours.

44. A toothpaste comprising the glass composition according to any one of claims 1 to 43.

45. A toothpaste comprising the glass composition according to any one of claims 38 to 42, wherein the toothpaste includes a sufficient amount of the glass composition to result in about 1,000 ppm to about 1,500 ppm of the fluoride.

46. A prophylaxis paste comprising the glass composition according to any one of claims 1 to 43.

47. A prophylaxis paste comprising the glass composition according to any one of claims 38 to 42, wherein the toothpaste includes a sufficient amount of the glass composition to result in about 1,000 ppm to about 1,500 ppm of the fluoride.

48. A tooth varnish comprising the glass composition according to any one of claims 1 to 43.

49. A tooth varnish comprising the glass composition according to any one of claims 38 to 42, wherein the toothpaste includes a sufficient amount of the glass composition to result in about 1,000 ppm to about 1,500 ppm of the fluoride.

50. Use of the toothpaste according to claim 44 or 45 to at least temporarily reduce pain associated with sensitive teeth.

51. Use of the prophylaxis paste according to claim 46 or 47 to at least temporarily reduce pain associated with sensitive teeth.

52. Use of the tooth varnish according to claim 48 or 49 to at least temporarily reduce pain associated with sensitive teeth.

53. A method of at least temporarily reducing, in an individual, pain associated with sensitive teeth, the method comprising applying:
the toothpaste according to claim 44 or 45,
the prophylaxis paste according to claim 46 or 47, or
the tooth varnish according to claim 48 or 49,
to dentin in the individual.

54. A glass composition according to any one of claims 1 to 43 for desensitizing dentin.

55. The glass composition for desensitizing dentin according to claim 54 for temporarily reducing pain associated with sensitive teeth.

56. A dentin-desensitizing composition comprising:
(i) the glass composition according to any one of claim 1 to 43, 54 or 55; and
(ii) a water-free, orally-compatible carrier.

57. The dentin-desensitizing composition according to claim 56 wherein the orally-compatible carrier is a mouthwash.

58. The dentin-desensitizing composition according to claim 56 wherein the orally-compatible carrier is formulated to mix with a mouthwash.

59. The dentin-desensitizing composition according to claim 56 wherein the orally-compatible carrier is an orally-compatible viscous carrier.

60. The dentin-desensitizing composition according to claim 59 wherein the orally-compatible viscous carrier has a viscosity from about 100 cP at 30° C. to about 150,000 cp at 30° C.

61. The dentin-desensitizing composition according to claim 59 wherein the orally-compatible viscous carrier is a toothpaste, a dental gel, a prophylaxis paste, a tooth varnish, or a bonding agent.

62. The dentin-desensitizing composition according to any one of claims 56 to 61, wherein:
the glass composition is the glass composition according to any one of claims 38 to 43; and
the dentin-desensitizing composition includes a sufficient amount of the glass composition to result in about 1,000 ppm to about 1,500 ppm of fluoride.

63. A glass composition comprising:
fluoride, which is provided as from about 5 mol % to about 10 mol % of CaF₂, SnF₂, NaF, KF, or any combination thereof, such as about 5 mol % of CaF₂ or about 10 mol % NaF; and
from about 90 mol % to about 95 mol % of a combination of B₂O₃, Na₂O, MgO, and CaO;
wherein the boron, the magnesium, the combination of Na and any K, and the combination of Ca and any Sn in the glass composition are present in elemental ratios of about 20: about 4: about 6: about 3, respectively,
wherein the glass composition loses at least 5 mass % within 24 hours when exposed to a buffered saline solution, and
wherein the glass composition is a particulate material that comprises particles that are from about 1 to about 50 μm in size.

64. The glass composition according to claim 63, comprising:
about 50 mol % B₂O₃,
about 15 mol % Na₂O,
about 20 mol % MgO,
about 10 mol % CaO, and
about 5 mol % NaF, KF, CaF₂, SnF₂, or any combination thereof.

65. The glass composition according to claim 63 or 64, wherein at least 75% of the particles are smaller than 50 μm in size.

66. The glass composition according to claim 63 or 64, wherein at least 85% of the particles are smaller than 50 μm in size.

67. The glass composition according to claim 63 or 64, wherein at least 95% of the particles are smaller than 50 μm in size.

68. The glass composition according to any one of claims 63 to 67, wherein at least 5% of the particles are smaller than 7 μm in size.

69. The glass composition according to any one of claims 63 to 67, wherein:
at least 5% of the particles are smaller than 35 μm in size,
at least 5% of the particles are smaller than 15 μm in size, and
at least 5% of the particles are smaller than 7 μm in size.

70. The glass composition according to any one of claims 63 to 67, wherein:
at least 5% of the particles are from about 15 μm to about 35 μm in size,
at least 5% of the particles are from about 6 μm to about 15 μm in size, and
at least 5% of the particles are from about 3 μm to about 7 μm in size.

71. The glass composition according to claim 63 or 64, wherein:
10% of the particles are smaller than 5 μm in size,
50% of the particles are smaller than 15 μm in size, and
90% of the particles are smaller than 30 μm in size.

72. The glass composition according to any one of claims 63 to 71, wherein the glass composition comprises about 5 mol % of CaF₂.

73. A toothpaste comprising the glass composition according to any one of claims 63 to 72.

74. A toothpaste comprising the glass composition according to any one of claims 62 to 71, wherein the toothpaste includes a sufficient amount of the glass composition to result in about 1,000 ppm to about 1,500 ppm of the fluoride.

75. A prophylaxis paste comprising the glass composition according to any one of claims 63 to 72.

76. A prophylaxis paste comprising the glass composition according to any one of claims 63 to 72, wherein the toothpaste includes a sufficient amount of the glass composition to result in about 1,000 ppm to about 1,500 ppm of the fluoride.

77. A tooth varnish comprising the glass composition according to any one of claims 63 to 72.

78. A tooth varnish comprising the glass composition according to any one of claims 63 to 72, wherein the toothpaste includes a sufficient amount of the glass composition to result in about 1,000 ppm to about 1,500 ppm of the fluoride.

79. The glass composition according to any one of claims 63 to 72 for desensitizing dentin.

80. A dentin-desensitizing composition comprising:
(i) the glass composition according to any one of claims 63 to 72; and
(ii) a water-free, orally-compatible carrier.

81. The dentin-desensitizing composition according to claim 80 wherein the orally-compatible carrier is a mouthwash.

82. The dentin-desensitizing composition according to claim 80 wherein the orally-compatible carrier is formulated to mix with a mouthwash.

83. The dentin-desensitizing composition according to claim 80 wherein the orally-compatible carrier is an orally-compatible viscous carrier.

84. The dentin-desensitizing composition according to claim 83 wherein the orally-compatible viscous carrier has a viscosity from about 100 cP at 30° C. to about 150,000 cp at 30° C.

85. The dentin-desensitizing composition according to claim 83 wherein the orally-compatible viscous carrier is a toothpaste, a dental gel, a prophylaxis paste, a tooth varnish, or a bonding agent.

86. The dentin-desensitizing composition according to any one of claims 80 to 85, wherein:
the dentin-desensitizing composition includes a sufficient amount of the glass composition to result in about 1,000 ppm to about 1,500 ppm of the fluoride.

87. A glass comprising:
from about 50 mol % to about 95 mol % of B₂O₃;
from about 5 mol % to about 50 mol % of one or more glass components selected from the group consisting of: Li₂O, Rb₂O, K₂O, Na₂O, SrO, CaO, MgO, and ZnO;
0 mol % of CuO;
less than 0.1 mol % of BaO; and
less than 0.1 mol % of P₂O₅;
wherein the glass comprises less than 30 mol % of Rb₂O, and
wherein the glass composition is at least a quaternary system.

88. A glass comprising:
fluoride, which is provided as from about 5 mol % to about 10 mol % of CaF₂, SnF₂, NaF, KF, or any combination thereof; and
from about 90 mol % to about 95 mol % of a combination of B₂O₃, Na₂O, MgO, and CaO, wherein the boron, the magnesium, the combination of Na and any K, and the combination of Ca and any Sn in the glass composition are present in elemental ratios of about 20: about 4: about 6: about 3, respectively.

89. The glass according to claim 88, comprising:
about 50 mol % B₂O₃,
about 15 mol % Na₂O,
about 20 mol % MgO,
about 10 mol % CaO, and
about 5 mol % NaF, KF, CaF₂, SnF₂, or any combination thereof.

90. The glass according to claim 89, comprising about 5 mol % of CaF₂.

91. The glass according to claim 88, comprising about 10 mol % NaF.

92. A bulk glass for preparing the glass composition according to any one of claims 1 to 43 and 63 to 72.
